(19) United States
(12) Patent Application Publication
(10) Pub. No.: US 2026/0007390 A1
(43) Pub. Date: Jan. 8, 2026

(54) TRANSMISSION AND RECEPTION DEVICE, ULTRASONIC PROBE, AND ULTRASONIC DIAGNOSTIC APPARATUS

(71) Applicant: KONICA MINOLTA, INC., Tokyo (JP)

(72) Inventors: Yuta NAKAYAMA, Ichikawa-shi (JP); Kenji SUZUKI, Tokyo (JP)

(73) Assignee: KONICA MINOLTA, INC., Tokyo (JP)

(21) Appl. No.: 19/247,954

(22) Filed: Jun. 24, 2025

(30) Foreign Application Priority Data
Jul. 3, 2024 (JP) 2024-107122

(51) Int. Cl. A61B 8/00; A61B 8/14; B06B 1/02; B06B 1/06

(52) U.S. Cl. CPC A61B 8/4494; A61B 8/14; B06B 1/0215; B06B 1/0622; B06B 1/0666; B06B 2201/76

(57) ABSTRACT

Disclosed is a transmission and reception device including: a plurality of PMUT sections in which each PMUT section includes one or more PMUTs on a CMOS circuit; and the CMOS circuit. The CMOS circuit includes: a wave transmitter that switches a voltage between a high voltage and a low voltage and applies the switched voltage to one or both of an upper electrode and a lower electrode of the PMUT section at a time of transmission of an ultrasound; and a series connector that connects the PMUT sections in series in an arbitrary number of series and connects the connected PMUT sections between a wave receiver and a common potential at a time of reception of an ultrasound, the wave receiver processing a reception signal of the connected PMUT sections.

### CROSS-REFERENCE TO RELATED APPLICATION

The entire disclosure of Japanese Patent Application No. 2024-107122, filed on Jul. 3, 2024, including description, claims, drawings and abstract is incorporated herein by reference.

### BACKGROUND OF THE INVENTION

### Technical Field

The present disclosure relates to a transmission and reception device, an ultrasonic probe, and an ultrasonic diagnostic apparatus.

### Description of Related Art

In ultrasonic diagnosis, the state of a heart or a fetus is obtained as an ultrasound image by a simple operation of bringing an ultrasonic probe into contact with the body surface or the inside of the body cavity of a subject of a patient. In addition, since the ultrasonic diagnosis is highly safe, the inspection can be repeatedly performed, ultrasound image data is obtained by transmitting ultrasound from an ultrasonic probe including piezoelectric elements to a subject, receiving the reflected ultrasound, and performing various processing on the reception signal.

In addition, an ultrasonic probe using a piezoelectric micromachined ultrasonic transducer (PMUT) is known.

The PMUT is a transducer based on bending motion of a thin film coupled to a piezoelectric thin film by micro electro mechanical systems (MEMS). For example, an ultrasonic probe is known in which electrical impedance is changed by switching wiring of transmission and reception elements of a PMUT between transmission and reception of ultrasound (see Japanese Patent No. 6405737).

There is also known an ultrasonic diagnostic apparatus including an AFE (Analog Front End) having an LNA (Low Noise Amplifier), a VGA (Variable Gain Amplifier), and an ADC (Analog-to-Digital Converter) (see Non Patent Literature 1). Non Patent Literature: Analog Devices. “Medical Ultrasound”. [online]. Analog Devices. [retrieved on Dec. 21, 2023]. Internet (URL: https://www.analog.com/jp/applications/markets/healthcare-pavilion-home/imaging/medical-ultrasound.html)).

The VGA is an amplifier for time gain compensation (TGC). This ultrasonic diagnostic apparatus has a memory in a field programmable gate array (FPGA) outside the AFE, and forms a reception beam former (BF).

A configuration in which a PMUT is applied to the AFE of the above-described Non Patent Literature will be considered. The PMUT is capable of impedance control by series-parallel connection. Therefore, it is possible to reduce a noise request to the LNA necessary for reception by the AFE. Therefore, the first power consumption of the AFE is the VGA of the TGC.

In particular, multi-channeling is expected in a PMUT on CMOS (Complementary Metal Oxide Semiconductor) system.

For this reason, the power consumption of the TGC required for the number of channels becomes a bottleneck for realizing a high sensitivity and high dynamic range system.

### SUMMARY OF THE INVENTION

It is an object of the present disclosure to achieve low power consumption and a high dynamic range in TGC.

To achieve at least one of the abovementioned objects, according to an aspect of the present invention, transmission and reception device reflecting one aspect of the present invention comprises:
a plurality of PMUT sections in which each PMUT section includes one or more PMUTs on a CMOS circuit; and
the CMOS circuit, wherein
the CMOS circuit includes:
   a wave transmitter that switches a voltage between a high voltage and a low voltage and applies the switched voltage to one or both of an upper electrode and a lower electrode of the PMUT section at a time of transmission of an ultrasound; and
   a series connector that connects the PMUT sections in series in an arbitrary number of series and connects the connected PMUT sections between a wave receiver and a common potential at a time of reception of an ultrasound, the wave receiver processing a reception signal of the connected PMUT sections.

To achieve at least one of the abovementioned objects, according to another aspect of the present invention, transmission and reception device reflecting one aspect of the present invention comprises:
an amplifier section configured to amplify a reception signal at a time of ultrasound reception from a PMUT section including one or a plurality of PMUTs;
a holder including a plurality of holding capacitance sections for holding the reception signal; and
a series capacitance section provided between the PMUT section and the holder and switchable together with the amplifier section as a path of the reception signal, wherein
a capacitance of the series capacitance section is smaller than or equal to a capacitance of each of the holding capacitance sections or is smaller than a total sum of capacities of the plurality of holding capacitance sections.

To achieve at least one of the abovementioned objects, according to another aspect of the present invention, ultrasonic probe reflecting one aspect of the present invention comprises the transmission and reception device according to any one of the above.

To achieve at least one of the abovementioned objects, according to another aspect of the present invention, ultrasonic diagnostic apparatus reflecting one aspect of the present invention comprises an ultrasonic probe comprising the ultrasonic probe described above.

### BRIEF DESCRIPTION OF THE DRAWINGS

The advantages and features provided by one or more embodiments of the invention will become more fully understood from the detailed description given hereinafter and the appended drawings which are given by way of illustration only, and thus are not intended as a definition of the limits of the present disclosure, and wherein:

FIG. 1 is a block diagram illustrating the functional configuration of the ultrasonic diagnostic apparatus according to the first embodiment of the present disclosure;

FIG. 2 is a schematic cross-sectional view showing the PMUT array and the CMOS circuit of the first embodiment;

FIG. 3 is a circuit diagram of the PMUT switching section and the wave receiver of the CMOS circuit of the first embodiment;

FIG. 4 is a diagram illustrating a switch switching state of the PMUT switching section at the time of ultrasound transmission (parallel positive sound pressure);

FIG. 5 is a diagram illustrating a switch changeover state of the PMUT switching section during ultrasound transmission (parallel negative sound pressure);

FIG. 6 is a diagram illustrating a switching state of the PMUT switching section according to the number of PMUT cells connected in series (3 series) at the time of ultrasound reception;

FIG. 7 is a diagram illustrating a switch switching state of the PMUT switching section according to the number of PMUT cells in series (two series) at the time of ultrasound reception;

FIG. 8 is a diagram illustrating a switching state of the PMUT switching section according to the number of PMUT cells in series (one series) at the time of ultrasound reception;

FIG. 9 is a diagram showing signal levels of a conventional PMUT switching section, an LNA, and a VGA with respect to depth or time;

FIG. 10 is a diagram illustrating signal levels of the PMUT switching part and the wave receiver of the first and second embodiment with respect to depth or time;

FIG. 11 is a diagram illustrating power consumption of amplifiers of a conventional PMUT switching section, an LNA, and a VGA, and the PMUT switching section and the wave receiver of the first and second embodiment, with respect to depth or time;

FIG. 12 is a diagram illustrating switch switching states of the amplification section and the holder of the time phase 0 at the time of the standard amplitude;

FIG. 13 is a diagram illustrating a switching state of the amplification section and the holder of the time phase 1 at the time of the standard amplitude;

FIG. 14 is a diagram illustrating a switching state of the amplification section and the holder of the time phase 2 at the time of the standard amplitude;

FIG. 15 is a diagram illustrating a switching state of the amplification section and the holder of the time phase 0 at the time of ½ amplitude;

FIG. 16 is a diagram illustrating a switching state of the amplification section and the holder of the time phase 1 at the time of ½ amplitude;

FIG. 17 is a diagram illustrating a switching state of the amplification section and the holder of the time phase 2 at the time of ½ amplitude;

FIG. 18 is a diagram illustrating a switch changeover state of the amplification section and the holder of a time phase 0 at the time of a low dynamic range;

FIG. 19 is a diagram illustrating a switch changeover state of the amplification section and the holder of a time phase 1 at the time of a low dynamic range;

FIG. 20 is a diagram illustrating a switching state of the amplification section and the holder of the time phase 2 at the time of a low dynamic range;

FIG. 21 is a diagram illustrating a switch changeover state of the amplification section and the holder of a time phase 0 at the time of a high dynamic range;

FIG. 22 is a diagram illustrating a switching state of the amplification section and the holder of a time phase 1 at the time of a high dynamic range;

FIG. 23 is a diagram illustrating a switching state of the holder and the capacitance switching section of a time phase 2 at the time of a high dynamic range;

FIG. 24 is a schematic cross-sectional view illustrating a PMUT array and a CMOS circuit of a first modification example;

FIG. 25 is a circuit diagram illustrating a PMUT switching section of a second modification example; and

FIG. 26 is a circuit diagram illustrating a PMUT switching section and a wave receiver of a CMOS circuit of a third modification example.

### DETAILED DESCRIPTION

Advantages and features provided by one or more embodiment of the present disclosure will be more fully understood from the following detailed description and the accompanying drawings. However, these drawings are for illustration purposes only. Therefore, it is not intended to define the limits of the present disclosure. Hereinafter, first and second embodiment and first to third modification example of the present disclosure will be described with reference to the drawings. However, the scope of the present disclosure is not limited to the disclosed embodiment and modification example.

### First Embodiment

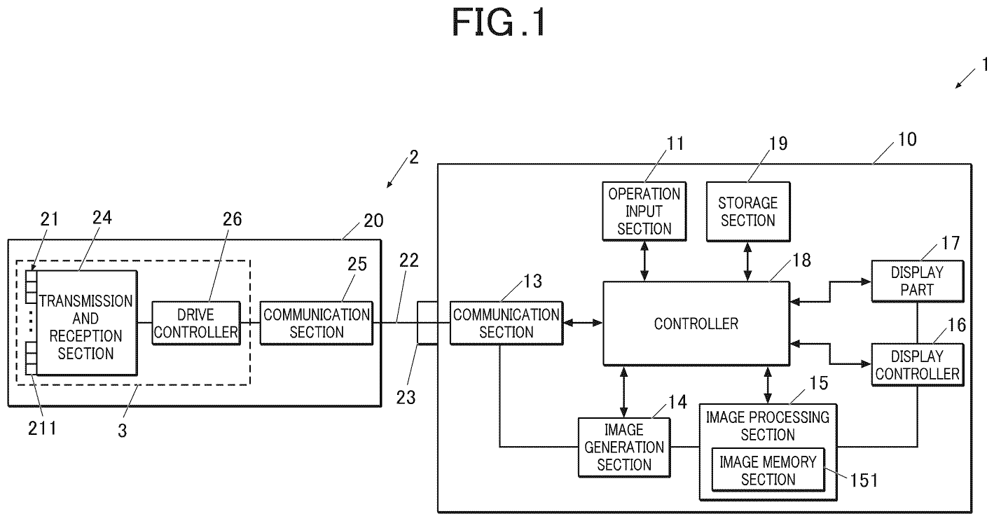

A first embodiment according to the present disclosure will be described with reference to FIGS. 1 to 11. First, the apparatus configuration of an ultrasonic diagnostic apparatus 1 according to the present embodiment will be described with reference to FIG. 1. FIG. 1 is a block diagram illustrating the functional configuration of an ultrasonic diagnostic apparatus 1 according to the present embodiment.

The ultrasonic diagnostic apparatus 1 is installed in a medical facility such as a hospital, is used by a user such as a doctor or a technician, and generates ultrasound image data of a subject such as a living body of a patient. As illustrated in FIG. 1, the ultrasonic diagnostic apparatus 1 includes an ultrasonic diagnostic apparatus main body 10 and an ultrasonic probe 2.

The ultrasonic probe 2 transmits ultrasound (transmitted ultrasound) into a subject and receives ultrasound reflected inside the subject (reflected ultrasound: echoes). The ultrasonic diagnostic apparatus main body 10 is connected to the ultrasonic probe 2. The ultrasonic diagnostic apparatus main body 10 transmits an electrical signal control signal to the ultrasonic probe 2 to cause the ultrasonic probe 2 to transmit a transmission ultrasound wave to the subject. Then, the ultrasonic diagnostic apparatus main body 10 receives a reception signal which is an electrical signal generated by the ultrasonic probe 2 according to the reflected ultrasound from the inside of the subject received by the ultrasonic probe 2. Then, the ultrasonic diagnostic apparatus main body 10 images the internal state of the subject as ultrasound image data based on the reception signal.

The ultrasonic probe 2 includes a head section 20, a cable 22, and a connector 23. The head section 20 includes a PMUT array 21, a transmission and reception section 24, a communication section 25, and a drive controller 26 (hardware processor).

The head section 20 is a tip part located on a tip side of the ultrasonic probe 2 to transmit and receive ultrasound, and has a PMUT array 21 formed by a plurality of PMUT cells 211 on its tip side. The PMUT cell 211 functions as a PMUT section. The ultrasonic probe 2 transmits and receives ultrasound with the PMUT array 21 side facing a subject. The PMUT cell 211 is formed of a MEMS, and is a piezoelectric element (transducer element) that transmits and receives ultrasound. It is assumed that the plurality of PMUT cells 211 are arranged, for example, in a one dimensional array shape in an azimuth direction (orientation direction, scanning direction), and the plurality of PMUT cells 320 are also arranged in an elevation direction perpendicular to the azimuth direction. Provided that a plurality of PMUT cells 211 may be arranged in a one dimensional array only in the azimuth direction. Further, the number of PMUT cells 211 in the PMUT array 21 can be arbitrarily set. Further, in the present embodiment, it is assumed that an electronic scan probe of a linear scanning system is used as the ultrasonic probe 2 to perform ultrasound scanning by the linear scanning system. However, the ultrasonic probe 2 may adopt other scanning methods such as a convex scanning method and a sector scanning method.

The cable 22 is a cable whose one end is electrically connected to the head section 20 and whose other end is electrically connected to the connector 23.

The connector 23 is a plug-type connector that is electrically connected to the cable 22 and is connected to the ultrasonic diagnostic apparatus main body 10. The connector 23 is detachably and electrically connected to a receptacle-type connector (not illustrated) of the ultrasonic diagnostic apparatus main body 10.

The transmission and reception section 24, as a transmitter, functions as a circuit that supplies a drive signal, which is an electrical signal, to the PMUT array 21 to generate ultrasound under the control of the controller 18 (drive controller 26). Furthermore, the transmission and reception section 24 includes, for example, a clock generating circuit, a delay circuit, and a pulse generating circuit. The clock generation circuit is a circuit that generates a clock signal that determines transmission timing and a transmission frequency of the drive signal. The delay circuit is a circuit that sets a delay time for each individual path corresponding to each PMUT cell and delays transmission of a drive signal by the set delay time. The delay circuit performs focusing of a transmission beam formed by transmission ultrasonic waves. The pulse generation circuit is a circuit for generating a pulse signal as a drive signal at a predetermined cycle, and performs switching for outputting drive power to each PMUT cell 211.

For example, the transmission and reception section 24 drives a continuous part of the plurality of PMUT cells 211 arranged in the ultrasonic probe 2 to generate transmission ultrasound waves. The transmission and reception section 24 performs scanning by shifting the PMUT cell to be driven in the azimuth direction every time the transmission ultrasonic wave is generated.

In addition, the transmission and reception section 24 functions as a receiver as a circuit that receives a reception signal which is an electrical signal from the PMUT array 21 according to the control of the controller 18 (drive controller 26). The transmission and reception section 24 includes, for example, an amplification section, a phasing section, an addition section, and an AD conversion section. The amplification section is a circuit that amplifies the reception signal at a preset amplification factor for each individual path corresponding to each PMUT cell 211. The phasing section is a circuit that gives a delay time to the amplified reception signal for each individual path corresponding to each PMUT cell to adjust the time phase (phasing). The adder is a circuit that adds the phased reception signals to generate a sound ray signal. The AD conversion section is a circuit that converts an analog sound ray signal into digital sound ray data. The transmission and reception section 24 transmits the sound ray data to the ultrasonic diagnostic apparatus main body 10 via the communication section 25. The execution order of the addition and the AD conversion may be reversed. Part of the transmission and reception section 24 will be described in detail later.

The drive controller 26 controls each part of the ultrasonic probe 2 mainly under the control of the controller 18. The drive controller 26 includes a central processing unit (CPU), a random access memory (RAM), a storage section, and a control circuit.

The communication section 25 is an interface that performs wired communication with the ultrasonic diagnostic apparatus main body 10 (communication section 13) via the cable 22 in a predetermined communication system. The drive controller 26 transmits and receives information to and from the ultrasonic diagnostic apparatus main body 10 via the communication section 25. Furthermore, under the control of the drive controller 26, the communication section 25 outputs, to the drive controller 26, the information for generating the drive signal received from the ultrasonic diagnostic apparatus main body 10. The communication section 25 transmits the sound ray data generated by the transmission and reception section 24 to the ultrasonic diagnostic apparatus main body 10 under the control of the drive controller 26.

The ultrasonic diagnostic apparatus main body 10 includes an operation input section 11, a communication section 13, an image generation section 14, an image processing section 15, a display controller 16, a display part 17, a controller 18, and a storage section 19.

The operation input section 11 accepts an operation input from a user. The operation input section 11 is, for example, an operation input section for inputting various image parameters for displaying a command instructing the start of diagnosis, ultrasound image data, and the like on the display part 17. The operation input section 11 includes various switches, buttons, a trackball, a mouse, a keyboard, and a touch pad, and outputs an operation signal to the controller 18. The operation input section 11 may include a touch screen that is provided on the display panel of the display part 17 and receives a touch input of the user.

The communication section 13 is an interface that performs wired communication with the ultrasonic probe 2 (communication section 25) via the cable 22 by a predetermined communication method. Controller 18 transmits and receives information to and from ultrasonic probe 2 via communication section 13. Furthermore, under the control of the controller 18, the communication section 13 outputs the sound ray data received from the ultrasonic probe 2 to the image generation section 14.

Note that the communication between the communication section 13 and 25 is not limited to wired communication. The communication sections 13 and 25 may be, for example, interfaces that perform communication by a wireless communication method such as ultra wide band (UWB).

In this configuration, the ultrasonic probe 2 includes a power supply constituted by a primary or secondary battery or the like. The power supply supplies source power to sections of the ultrasonic probe 2.

Under the control of the controller 18, the image generation section 14 performs envelope detection processing, logarithmic compression, and the like on the sound ray data from the communication section 13, and performs luminance conversion by adjusting the dynamic range and the gain. Thus, the image generation section 14 generates B (Brightness) mode image data including pixels having a luminance value as the received energy. That is, the B-mode image data represents an intensity of a reception signal by a brightness. The image generation section 14 may be configured to generate ultrasound image data in another image mode such as a color Doppler mode, in addition to the B-mode image data whose image mode is the B mode.

Under the control of the controller 18, the image processing section 15 performs image processing on the B-mode image data output from the image generation section 14 in accordance with various image parameters being set. The image processing section 15 further includes an image memory section 151 including a semiconductor memory, such as a dynamic random access memory (DRAM). The image processing section 15 stores the B-mode image data subjected to the image processing in the image memory section 151 in units of frames in accordance with the control of the controller 18. The image data in units of frames may be referred to as ultrasound image data or frame image data. The image processing section 15 sequentially outputs the image data generated as described above to the display controller 16 under the control of the controller 18.

Under the control of the controller 18, the display controller 16 converts the ultrasound image data received from the image processing section 15 into an image signal for display by performing coordinate conversion or the like, and outputs the image signal to the display part 17.

As the display part 17, a display device such as a liquid crystal display (LCD), a cathode-ray tube (CRT) display, an organic electronic luminescence (EL) display, an inorganic EL display, or a plasma display is applicable. Under the control of the controller 18, the display part 17 displays a still image or a moving image of the ultrasound image data on the display screen according to the image signal output from the display controller 16.

The controller 18 includes, for example, a CPU, a RAM, and a storage section. The controller 18 reads various programs stored in the storage section, develops the programs in the RAM, and controls each section of the ultrasonic diagnostic apparatus 1 in accordance with the developed programs. The storage section includes a non-volatile memory such as a semiconductor. The storage section stores a system program corresponding to the ultrasonic diagnostic apparatus 1, various processing programs executable on the system program, various data such as various tables, and the like. These programs are stored in the form of computer-readable program codes. The CPU sequentially executes operation according to the program code. The RAM forms a work area in which various programs executed by the CPU and data related to these program are temporarily stored.

In particular, the controller 18 receives operation information on ultrasound image display from the user via the operation input section 11. Controller 18 causes transmission and reception section 24 to generate a drive signal and cause PMUT cell 211 to output ultrasound, based on the operation information. The controller 18 causes the transmission and reception section 24 to receive the reception signal input from the PMUT cell 211 and generate sound ray data. The controller 18 causes the image generation section 14 to generate B-mode image data from the sound ray data. The controller 18 causes the display part 17 to display the B-mode image data as a B-mode image via the image processing section 15 and the display controller 16.

The storage section 19 includes a hard disk drive (HDD) and a solid state drive (SSD), and stores data such as ultrasound image data.

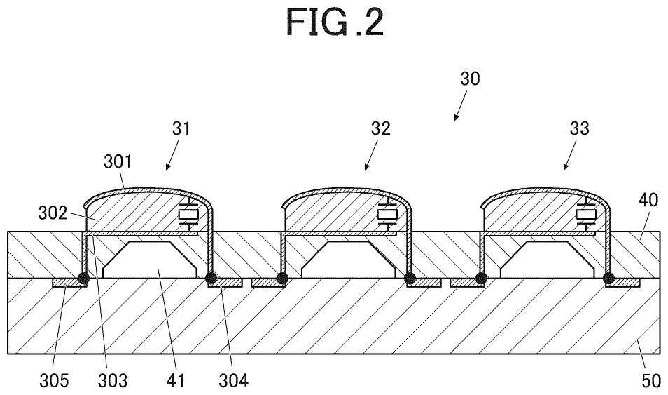

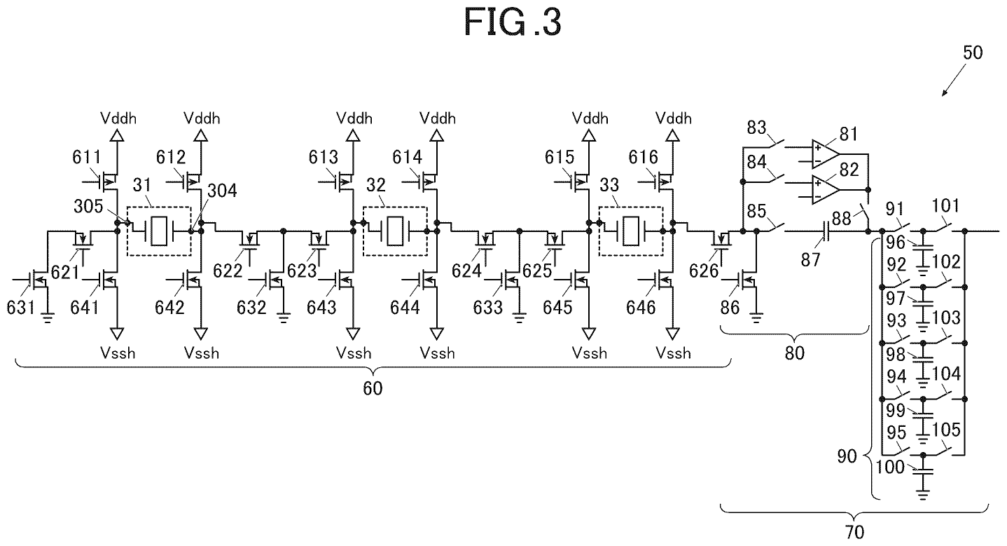

Next, detailed configurations of the PMUT array 21 and the transmission and reception section 24 will be described with reference to FIGS. 2 and 3. FIG. 2 is a schematic cross-sectional view showing the PMUT array 30 and the CMOS circuit 50. FIG. 3 is a circuit diagram of the PMUT switching section 60 and the wave receiver 70 of the CMOS circuit 50.

Here. FIGS. 2 and 3 representatively illustrate the configurations of the PMUT array 21 and the transmission and reception section 24 for one channel. In practice, the PMUT array 21 and the transmission and reception section 24 have a configuration of a plurality of channels.

As shown in FIG. 2, as an example of PMUT array 21 of ultrasonic probe 2. PMUT array 30 in which PMUT cells 31, 32, and 33 as PMUT cells 211 are arranged in order will be described. Here, for simplicity of explanation, the PMUT array 30 including three PMUT cells 31, 32, and 33 will be described. However, the number of PMUT cells 211 is not limited to this example.

The PMUT array 30 is a PMUT on CMOS in which PMUT cells 31, 32, and 33 are stacked on a CMOS circuit 50.

In each of PMUT cells 31, 32, and 33, MEMS part 40, lower electrode 303. PMUT cell main body 302, and upper electrode 301 are stacked in order from the lower side to the upper side. The upper electrode 301 is an electrode made of metal such as Pt disposed on an upper portion of the PMUT cell body 302. The lower electrode 303 is an electrode, such as metal Pt, disposed at a lower portion of the PMUT cell body 302. The PMUT cell body 302 is a main body section of a piezoelectric element, and is formed of a layer of lead zirconate titanate (P20) that is a piezoelectric material. In FIG. 2, the upper electrode 301, the PMUT cell main body 302, and the lower electrode 303 of each of the PMUT cells 31, 32, and 33 are illustrated in the form of a capacitor.

The MEMS part 40 is a layer of MEMS forming a thin film on which the upper electrodes 301, the PMUT cell main bodies 302, and the lower electrodes 303 of the PMUT cells 31, 32, and 33 are disposed. The MEMS section 40 is formed by stacking, for example, a SiO₂ layer, an Si layer, a SiO₂ layer, an Si layer, an SiO₂ layer, and a Ti layer in this order from the lower side to the upper side. The MEMS part 40 has a cavity part 41 which is an arch-shaped cavity. The PMUT cell bodies 302 of the PMUT cells 31, 32, and 33 are arranged on the cavity part 41 and are vibrated on the thin film (diaphragm).

Further, the MEMS part 40 of the PMUT cell 33 close to the wave receiver 70 may be used as the high frequency diaphragm, and the MEMS part 40 of the PMUT cell 31 distant from the wave receiver 70 may be used as the low frequency diaphragm. The high frequency diaphragm is a diaphragm that resonates at a high frequency. The low frequency diaphragm is a diaphragm that resonates at a low frequency. The PMUT cell 31 of the high frequency diaphragm is used in a shallow portion in which attenuation is small even at high frequencies at the time of ultrasound reception. The PMUT cell 33 of a low frequency diaphragm that reaches a deep portion is used in the deep portion. To increase effective sensitivity by providing a PMUT cell with depth-specific frequency characteristics.

A through silicon via (TSV) 304 is electrically connected to the upper electrode 301.

A lower electrode TSV305 is electrically connected to the lower electrode 303. The terminals TSV304 and 305 are electrically connected to the electrodes of the CMOS circuit 50 through the MEMS part 40, respectively. In FIG. 2, connection points between TSV304 and 305 and the electrodes of the CMOS circuit 50 are represented by large black circles.

The CMOS circuit 50 is a layer having a circuit including circuit elements such as a CMOS of the transmission and reception section 24. The PMUT array 30 is a PMUT on CMOS. Therefore, the parasitic capacitance of PMUT cells 31, 32, and 33 is extremely small. Further, the electrostatic capacitance of PMUT cells 31, 32, and 33 is sufficiently larger than the parasitic capacitance. Therefore, the number of connections between the PMUT cells 31, 32, 33 and the CMOS circuit 50 can be maximized.

As illustrated in FIG. 3, the CMOS circuit 50 includes a PMUT switching section 60 and a wave receiver 70. The wave receiver 70 includes an amplification section 80 and a holder 90).

The PMUT switching section 60) is a circuit that switches the connection of the PMUT cells 31, 32, and 33 at the time of ultrasound transmission and reception. The PMUT switching section 60 includes the PMUT cells 31, 32, and 33, the transmission pulsers 611, 612, 613, 614, 615, 616, 641, 642, 643, 644, 645, and 646, the series connection switches 621, 622, 623, 624, 625, and 626, and the GND switches 631, 632, and 633. The transmission pulsers 611 to 616 and 641 to 646 function as wave transmitters. The series connection switches 621 to 626 function as a series connector.

Here, circuit elements related to the PMUT cell 31 will be representatively described. The transmission pulser 611 is a P-channel MOS field effect transistor (PMOSFET) whose source is connected to a power supply of a high voltage Vddh and whose drain is connected to a TSV305. The high voltage Vddh is a power supply voltage higher than a GND potential that is a common potential.

The transmission pulser 612 is a PMOSFET whose source is connected to a power supply of a high voltage Vddh and whose drain is connected to a TSV304.

The series connection switch 621 is an N-channel MOS (NMOS) FET having a drain connected to the TSV305 and a source connected to the GND switch 631, series connection switch 6

An NMOSFET 22 has a drain connected to the TSV304 and a source connected to the GND switch 632.

The GND switch 631 is an NMOSFET whose drain is connected to the series connection switch 621 and whose source is connected to the GND.

The transmission pulser 641 is an NMOSFET whose drain is connected to the TSV305 and whose source is connected to a voltage source of the low voltage Vssh. The transmission pulser 611 forms a CMOS together with the transmission pulser 641. The transmission pulser 642 is an NMOSFET whose drain is connected to the TSV304 and whose source is connected to a power supply of a low voltage Vssh. The transmission pulser 612 forms a CMOS with the transmission pulser 642. The low voltage Vssh is a power supply voltage that is lower than the high voltage Vddh and the GND potential or equivalent to the GND potential.

The transmission pulsers 613, 614, 643, and 644 related to the PMUT cell 32 are similar to the transmission pulsers 611, 612, 641, and 642 related to the PMUT cell 31, respectively. The series connection switches 623 and 624 related to the PMUT cell 32 are similar to the series connection switches 621 and 622 related to the PMUT cell 31, respectively. The GND switch 632 related to the PMUT cell 32 is similar to the GND switch 631 related to the PMUT cell 31.

The transmission pulsers 615, 616, 645, and 646 related to the PMUT cell 33 are similar to the transmission pulsers 611, 612, 641, and 642 related to the PMUT cell 31, respectively. The series connection switches 625 and 626 related to the PMUT cell 33 are similar to the series connection switches 621 and 622 related to the PMUT cell 31, respectively. The GND switch 633 related to the PMUT cell 33 is similar to the GND switch 631 related to the PMUT cell 31.

In the transmission pulsers 611 to 616 and 641 to 646, a drive signal from the drive controller 26 is input to each gate, and switching is controlled. In the series connection switches 621 to 626 and the GND switches 631 to 633, a drive signal from the drive controller 26 is input to each gate, and switching is controlled.

The amplification section 80 is a sample and holder circuit for delaying and amplifying a current of the reception signal from the PMUT switching section 60. The amplification section 80 includes amplifiers 81 and 82, switches 83, 84, and 85, a GND switch 86, a capacitor 87, and a switch 88. The amplifiers 81 and 82 function as amplifier sections. The capacitor 87 functions as a series capacitance section.

The amplifier 81 is an operational amplifier for a high dynamic range. The power consumption of the amplifier 81 is higher than that of the amplifier 82. The output noise of the amplifier 81 is lower than that of the amplifier 82. The amplifier 82 is an operational amplifier with low power consumption. The amplifier 82 has low power consumption, but output noise is higher than that of the amplifier 81.

The switch 83 is a switch that turns on and off the input of the current from the PMUT switching section 60 to the amplifier 81 under the control of the drive controller 26. The switch 84 is a switch that turns on and off the input of the current from the PMUT switching section 60 to the amplifier 82 under the control of the drive controller 26. The switch 85 is a switch that turns on and off the input of the current from the PMUT switching section 60 to the capacitor 87 under the control of the drive controller 26.

The GND switch 86 is an NMOSFET whose drain is connected to the series connection switch 626 and whose source is connected to the GND. A drive signal from the drive controller 26 is input to the gate of the GND switch 86, and the GND switch 86 is controlled to be switched.

The capacitor 87 is a capacitor connected to the PMUT switching section 60 (the source of the series connection switch 626) in series and connected to the amplifiers 81 and 82 in parallel. The switches 83, 84, and 85 switch the connection destination of the PMUT switching section 60 to the capacitor 87 and the amplifiers 81 and 82. When the capacitor 87 is connected to the PMUT switching section 60, the capacitor 87 is charged with a reception signal from the PMUT switching section 60 and held.

An electric capacitance (referred to as a series capacitance C) of the capacitor 87 is less than or equal to an electric capacitance (referred to as a holding capacitance Cs) of each of the capacitors 96 to 100 of the holder 90. Alternatively, the series capacitance C of the capacitor 87 is set to be less than the total sum of the respective holding capacitances Cs of the capacitors 96 to 100. More specifically, the holding capacitance Cs is 2 [pF] or less. Since the output impedance of the amplifiers 81 and 82 is sufficiently smaller than the holding capacitance of the holder 90, the amplitude of the reception signal does not change, but the magnitude of the noise floor changes with the magnitude of the holding capacitance.

Further, the series capacitance C is equal to or less than the input capacitance of the amplifiers 81 and 82. Therefore, it is possible to suppress the amplitude fluctuation when the amplifier 81 or 82 and the capacitor 87 are switched as the connection destination of the PMUT switching section 60.

The switch 88 is a switch that turns on and off the output of the current from the amplifier 81 or 82 under the control of the drive controller 26.

The holder 90 is a circuit that holds an input current from the amplification section 80, switches the degree of amplification according to the depth of an ultrasound image, and outputs the input current. The holder 90 includes the switches 91, 92, 93, 94, and 95, the capacitors 96, 97, 98, 99, and 100, and the switches 101, 102, 103, 104, and 105.

The switch 91 is a switch connected to the amplification section 80 (the capacitor 87 and the switch 88) and the capacitor 96. Similarly, the switch 92 is a switch connected to the amplification section 80 and the capacitor 97. The switch 93 is a switch connected to the amplification section 80 and the capacitor 98. The switch 94 is a switch connected to the amplification section 80 and the capacitor 99. The switch 95 is a switch connected to the amplification section 80 and the capacitor 100. Under the control of the drive controller 26, the switches 91 to 95 turn on and off the input of the reception signals from the amplification section 80 to the capacitors 96 to 100, respectively.

The capacitor 96 is a capacitor whose one end is connected to the switches 91 and 101 and whose other end is connected to the GND. Similarly, the capacitor 97 is a capacitor whose one end is connected to the switches 92 and 102 and whose other end is connected to the GND. The capacitor 98 is a capacitor whose one end is connected to the switches 93 and 103 and whose other end is connected to the GND. The capacitor 99 is a capacitor whose one end is connected to the switches 94 and 104 and whose other end is connected to the GND. The capacitor 100 is a capacitor having one end connected to the switches 95 and 105 and the other end connected to the GND. The capacitors 96 to 100 each charge, hold, and output the reception signal from the amplification section 80.

The switch 101 is a switch connected to the capacitor 96 and the conversion section (or the adder) of the transmission and reception section 24. Similarly, the switch 102 is a switch connected to the capacitor 97 and the AD conversion section of the transmission and reception section 24. The switch 103 is a switch connected to the capacitor 98 and the conversion section of the transmission and reception section 24. The switch 104 is a switch connected to the capacitor 99 and the conversion section of the transmission and reception section 24. The switch 105 is a switch connected to the capacitor 100 and a conversion section of the transmission and reception section 24. The switches 101 to 105 turn on and off the output of the reception signals from the capacitors 96 to 100, respectively, under the control of the drive controller 26.

Note that in FIGS. 2 and 3, each of the PMUT cells 31 to 33 is a single cell of the PMUT section including one PMUT cell, but is not limited thereto. Each of the PMUT sections may be configured to include a plurality of PMUTs. The plurality of PMUTs of each PMUT section are configured to be connected in series or in parallel by upper electrodes and lower electrodes in the MEMS.

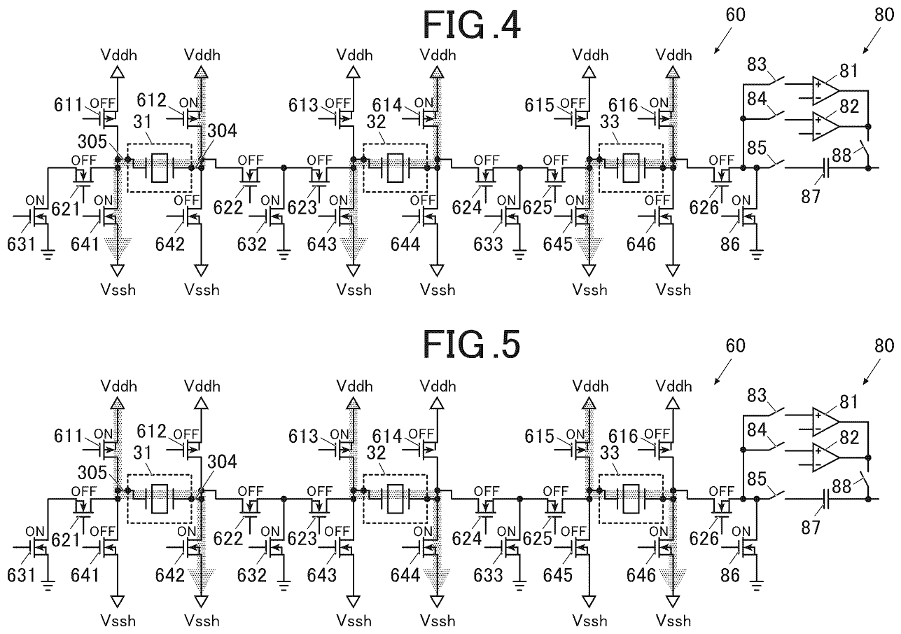

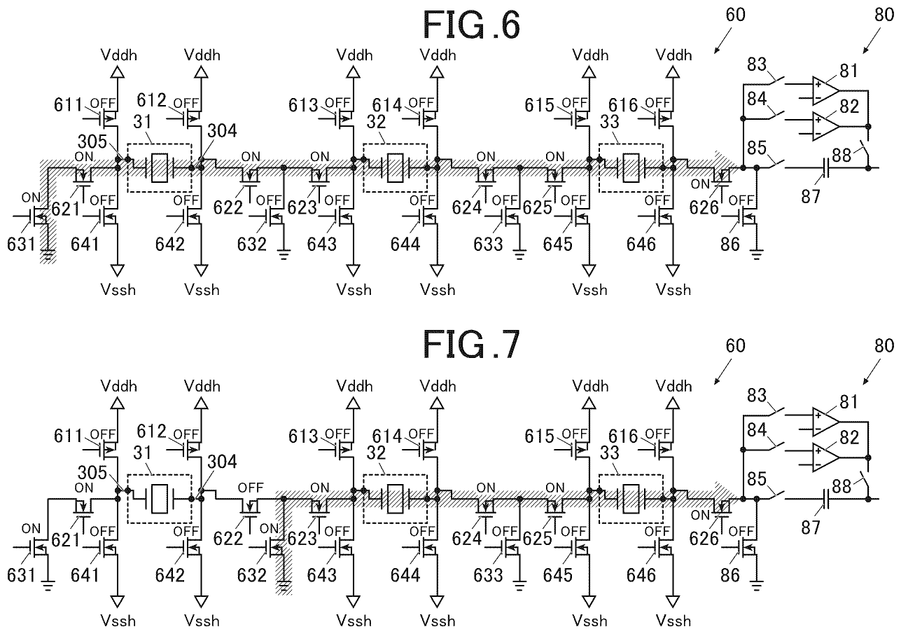

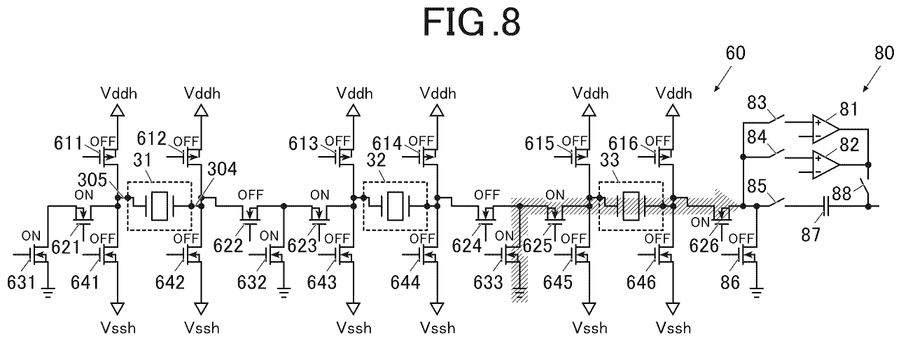

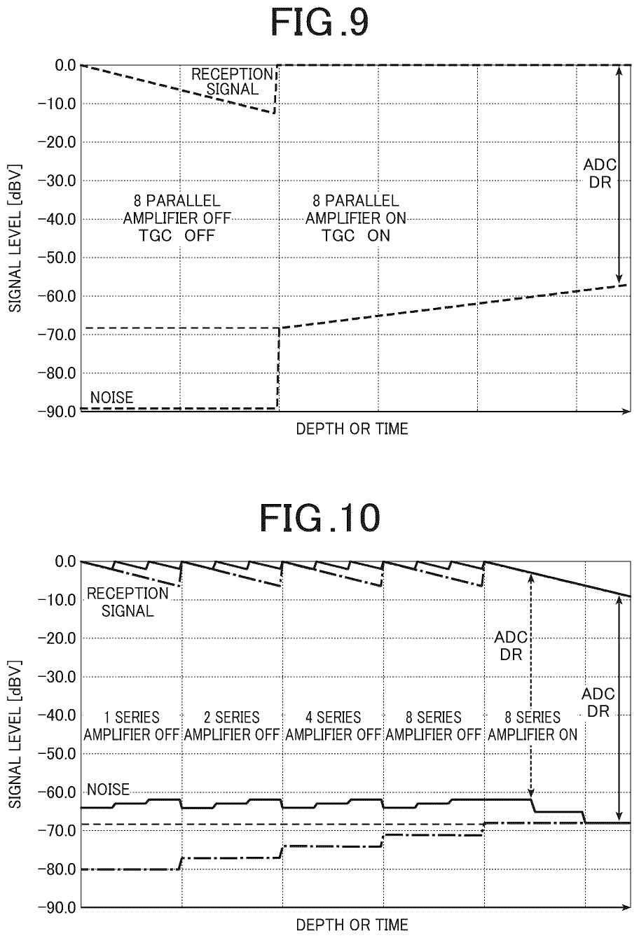

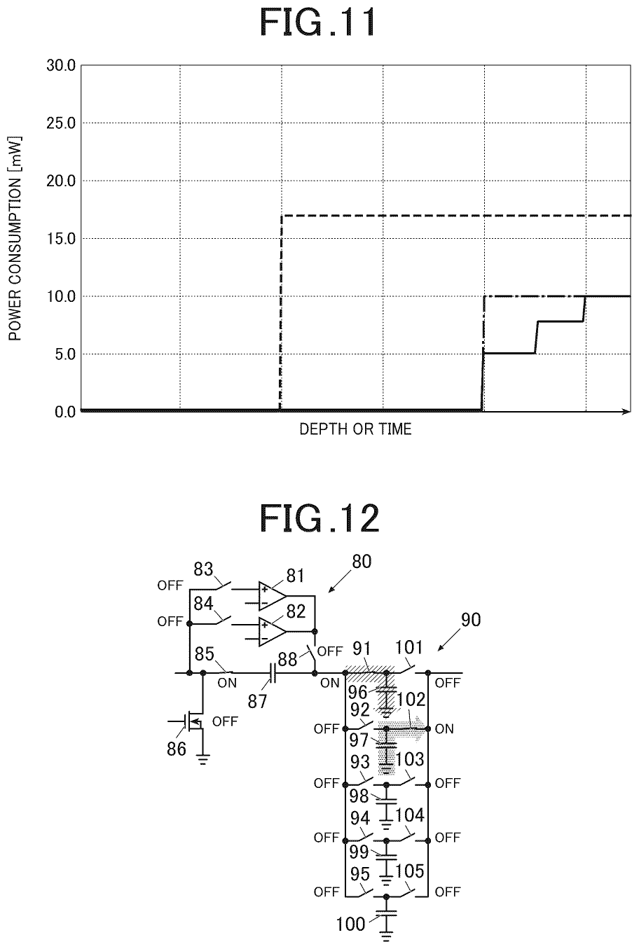

Next, operation of the PMUT switching section 60 and the wave receiver 70 will be described with reference to FIGS. 4 to 11. FIG. 4 is a diagram illustrating a switching state of the PMUT switching section 60 during ultrasound transmission (parallel positive sound pressure). FIG. 5 is a diagram illustrating a switching state of the PMUT switching section 60 at the time of ultrasound transmission (parallel negative sound pressure). FIG. 6 is a diagram illustrating a switching state of the PMUT switching section 60 according to the number of PMUT cells connected in series (three series) at the time of ultrasound reception. FIG. 7 is a diagram illustrating a switching state of the PMUT switching section 60 according to the number of PMUT cells connected in series (two series) at the time of ultrasound reception. FIG. 8 is a diagram illustrating a switching state of the PMUT switching section 60 according to the number of PMUT cells connected in series (one series) at the time of ultrasound reception. FIG. 9) is a diagram illustrating signal levels of the PMUT switching section, the LNA, and the VGA in the related art with respect to depth or time. FIG. 10 is a diagram illustrating signal levels of the PMUT switching section 60 and the wave receiver 70 of the present embodiment and the second embodiment with respect to depth or time. FIG. 11 is a diagram illustrating power consumption of amplifiers of the PMUT switching section, the LNA, and the VGA of the related art, and the PMUT switching section 60 and the wave receiver 70 of the first and second embodiment, with respect to depth or time.

Note that here, the operation of the PMUT switching section 60 and the wave receiver 70 for one channel will be described as a representative.

Switching of the switch of the PMUT switching section 60 at the time of ultrasound transmission will be described with reference to FIG. 4 and FIG. 5. At the time of ultrasound transmission, the transmission pulser full-bridge-drives the PMUT cells in parallel. As illustrated in FIG. 4, a case where positive sound pressures are obtained in parallel from the PMUT cells 31, 32, and 33 at the time of ultrasound transmission will be considered. The GND switches 631 to 633, and 86 are turned on. The series connection switches 621 to 626 are turned off. In a case where a voltage for positive sound pressure is applied to the PMUT cell 31 in this state, the transmission pulsers 611 and 642 are turned off. The transmission pulsers 612 and 641 are turned on.

Then, a current flows in a direction of an arrow drawn on the PMUT cell 31 of FIG. 4. That is, the upper electrode 301 of the PMUT cell 31 is set to the high voltage Vddh. The lower electrode 303 of the PMUT cell 31 is set to the low voltage Vssh. Thus, a positive sound pressure is obtained from the PMUT cell 31. Similarly, a positive sound pressure is also obtained from the PMUT cells 32 and 33 in parallel.

As illustrated in FIG. 5, a case where negative sound pressures are obtained in parallel from the PMUT cells 31, 32, and 33 at the time of ultrasound transmission will be considered. The GND switches 631 to 633, and 86 are turned on. The series connection switches 621 to 626 are turned off. In a case where a voltage for negative sound pressure is applied to the PMUT cell 31 in this state, the transmission pulsers 611 and 642 are turned on. The transmission pulsers 612 and 641 are turned off.

Then, a current flows in a direction of an arrow drawn on the PMUT cell 31 of FIG. 5. That is, the upper electrode 301 of the PMUT cell 31 is set to the low voltage Vssh. The lower electrode 303 of the PMUT cell 31 is set to the high voltage Vddh. Thus, a negative sound pressure is obtained from the PMUT cell 31. Similarly, a negative sound pressure is also obtained from the PMUT cells 32 and 33 in parallel with the PMUT cell 31. By repeating the driving of the PMUT cells 31 to 33 with the positive sound pressure and the negative sound pressure, ultrasound is transmitted from the PMUT cells 31 to 33.

With the configuration and driving of the full bridge of the PMUT cells 31 to 33, it is possible to obtain a high sound pressure with a half circuit voltage as compared with a configuration and driving of a half bridge to be described later. In addition, in the full-bridge driving, it is not necessary to eliminate a difference between a resistance of an NMOSFET and a resistance of a PMOSFET in the CMOS, and it is possible to improve the positive-negative symmetry. Therefore, transmission linearity and harmonic performance can be improved by the full-bridge driving. The transmission linearity is an error of a linear output with respect to an ideal curve. Harmonic performance is the accuracy of an ultrasound image due to harmonics.

Switching of the switch of the PMUT switching section 60 at the time of ultrasound reception will be described with reference to FIG. 6 to FIG. 8. At the time of ultrasound reception, a reception signal is obtained from a desired number of PMUT cells connected in series. As shown in FIG. 6, a case of three series reception in which reception signals are obtained in series from PMUT cells 31, 32, and 33 at the time of ultrasound reception will be considered. The transmission pulsers 611 to 616 and 641 to 646 are turned off.

The GND switch 631 is turned on. The GND switches 632, 633, and 86 are turned off. The series connection switches 621 to 626 are turned on. Then, a current flows in the direction of arrows drawn on PMUT cells 31, 32, and 33 in FIG. 6. That is, a reception signal of the ultrasound received by the PMUT cells 31, 32, and 33 connected in series is output to the amplification section 80.

As shown in FIG. 7, a case of two series reception in which reception signals are obtained in series from PMUT cells 32 and 33 at the time of ultrasound reception will be considered. The transmission pulsers 611 to 616 and 641 to 646 are turned off.

The GND switches 631 and 632 are turned on. The GND switches 633 and 86 are turned off. The series connection switch 622 is turned off. The series connection switches 621 and 623 to 626 are turned on. Then, a current flows in a direction of an arrow drawn on the PMUT cells 32 and 33 of FIG. 7. That is, the signal of the ultrasound received by the PMUT cells 32 and 33 connected in series is output to the amplification section 80. As illustrated in FIG. 8, a case of one series reception in which a reception signal is obtained from the PMUT cell 33 at the time of ultrasound reception will be considered. The transmission pulsers 611 to 616 and 641 to 646 are turned off.

The GND switches 631, 632, and 633 are turned on. The GND switch 86 is turned off. The series connection switches 622 and 624 are turned off. The series connection switches 621, 623, 625, and 626 are turned on. Then, a current flows in a direction of an arrow drawn on the PMUT cell 33 of FIG. 8. That is, the reception signal of the ultrasound received by the PMUT cell 33 is output to the amplification section 80.

In this way, the series connection switches 621 to 626 can connect an arbitrary number of PMUT cells in series and drop them to the GND (switch between GND termination and through) during the reception operation. A PMUT cell close to the amplification section 80 as a reception circuit is also used in a shallow portion. Therefore, the PMUT cell 33 close to the amplification section 80 is allocated for high frequency. Similarly, the far PMUT cell 31 is assigned for low frequencies. Similarly, the PMUT cell 32 at the intermediate position is allocated for an intermediate frequency. By these assignments, it is also possible to provide the PMUT switching section 60 with frequency characteristics for each depth.

Next, with reference to FIGS. 9 to 11, characteristics of the PMUT switching section, the LNA, and the VGA of the related art and the PMUT switching section 60 and the wave receiver 70 of the present embodiment at the time of ultrasonic wave reception will be described. First, with reference to FIG. 9, characteristics of a conventional PMUT switching section will be described. FIG. 9 illustrates signal levels [dBV] of reception signal and noise of the PMUT switching section, the LNA, and the VGA of the related art with respect to depth or time. This noise is temperature-induced noise, and the same applies to the following noise. A reception signal and noise of the conventional PMUT switching section are indicated by a broken line.

In the conventional PMUT switching section, the series connection of the PMUT cells is not switched, and a reception signal of the PMUT cells connected in parallel is generated at the time of ultrasound reception. As an example, it is assumed that the conventional PMUT switching section includes eight PMUT cells. It is assumed that the LNA and the VGA of the TGC are connected to the subsequent stage of the conventional PMUT switching section.

In addition, the conventional PMUT switching section is included in the CMOS section disposed immediately below the PMUT cell. In order to place the CMOS part immediately below the PMUT cell, it is necessary to suppress heat generation and suppress temperature-induced noise. For this reason, a low-noise (high power consumption) amplifier cannot be placed at the subsequent stage of the conventional PMUT switching section. Therefore, there is no choice but to adopt a feasible amplifier.

As illustrated in FIG. 9, in the conventional PMUT switching section, the amplifiers (LNA and VGA) in the subsequent stage are turned off in the shallow portion of the ultrasound image. This is to prevent the reception signal from being saturated because the level of the reception signal in the shallow portion is high. A reception signal level of a conventional PMUT switching section at the time of ultrasound reception decreases due to attenuation in accordance with an increase in depth or time. The noise does not change. Therefore, at a predetermined depth or time, the LNA and the VGA of the subsequent stage in the PMUT switching section of the related art are turned on. Then, although the reception signal level is increased to 0) [dBV], the noise also increases according to an increase in the depth or the time. Therefore, a dynamic range (DR) in an AD conversion section (ADC) in a subsequent stage of the PMUT switching section, the LNA, and the VGA in the related art is limited. The dynamic range is a signal level of reception signal-noise.

Next, characteristics of the PMUT switching section 60 and the wave receiver 70 of the present embodiment at the time of ultrasonic wave reception will be described with reference to FIGS. 10 and 11. FIG. 10 illustrates a signal level [dBV] of the reception signal and the temperature-induced noise of the PMUT switching section 60 and the wave receiver 70 of the present embodiment with respect to depth or time. The reception signal and noise of the PMUT switching section 60 and the wave receiver 70 of the present embodiment are indicated by chain lines. As an example, it is assumed that the PMUT switching section 60 has eight PMUT cells similar to the PMUT3133, and the number of series-connected PMUT cells can be switched.

As shown in FIG. 10, in the PMUT switching section 60) and the wave receiver 70 of the present embodiment, the amplifier of the amplification section 80 is turned off in the shallow portion of the ultrasound image. In the PMUT switching section 60 according to the present embodiment, the number of PMUT cells connected in series is switched to one (one in series). The reception signal levels of the PMUT switching section 60 and the wave receiver 70 according to the present embodiment decrease due to attenuation in accordance with an increase in depth or time. The noise does not change. Therefore, at a predetermined depth or time, in the PMUT switching section 60 of the present embodiment, the number of series PMUT cells is switched to two (two series). Then, the reception signal level increases to 0 [dBV]. However, the noise also increases by V 2 times.

The reception signal levels of the PMUT switching section 60 and the wave receiver 70 of the present embodiment decrease due to attenuation in accordance with a further increase in depth or time. The noise does not change. Therefore, at a predetermined depth or time, in the PMUT switching section 60 of the present embodiment, the number of series of the PMUT cells is switched to four (four series). Then, the reception signal level increases to 0 [dBV]. However, the noise also increases by V 2 times.

The reception signal levels of the PMUT switching section 60 and the wave receiver 70 of the present embodiment decrease due to attenuation in accordance with a further increase in depth or time. The noise does not change. Therefore, at a predetermined depth or time, the number of PMUT cells connected in series is switched to eight (eight in series) in the PMUT switching section 60 of the present embodiment. Then, the reception signal level increases to 0 [dBV]. However, the noise also increases by v 2 times.

The number of PMUT cells connected in series increases to the maximum (here, increased to 8). Then, the amplifier can be designed such that the noise of the PMUT switching section 60 and the wave receiver 70 according to the present embodiment becomes the same level as the noise level of the amplifier at a predetermined depth or time. At this time, in the wave receiver 70 of the present embodiment, the amplifier is switched on. In the examples of FIGS. 10 and 11, the amplifier 81 is turned on. Thus, the power consumption of the amplifier at the depth or lower can be reduced. Furthermore, by increasing the number of PMUT cells connected in series as the depth or the time increases, the reception signal level can be made substantially constant. At a greater depth, the reception signal level is increased by turning on the amplifier 81. In this way, the TGC operation can also be realized. The dynamic range in the PMUT switching section 60 and the A/D conversion section in the subsequent stage of the wave receiver 70 of the present embodiment is not changed even if the power consumption is reduced, compared to the configuration of the PMUT switching section, the amplifier, and the TGC of the related art.

Since the impedance of the wave receiver 70 is larger than that of the PMUT single cell, the voltage of the reception signal increases as the number of PMUT cells connected in series in the PMUT switching section 60 increases, and the PMUT switching section 60 has an appropriate point. Since aperture controls of similar concepts are performed in parallel, the impedance rather decreases in a deep portion, and therefore, there has been a problem that the minimum detection sensitivity is sacrificed particularly under a low power consumption limit.

FIG. 11 shows power consumption [mW] of amplifiers of a conventional PMUT switching section, an LNA, and a VGA, and the PMUT switching section 60 and the wave receiver 70 according to the present embodiment, with respect to depth or time. Power consumption of the configuration of the PMUT switching section, the amplifier, and the TGC according to the related art is indicated by a broken line. The power consumption of the PMUT switching section 60) and the wave receiver 70 according to the present embodiment is indicated by a chain line.

In the depth or the time of the PMUT switching section, the LNA, and the VGA of the related art, when the LNA and the VGA are turned off, the power consumption of the amplifier becomes 0 [mW]. In a case of a predetermined depth or time or more, the power consumption of the amplifier is greatly increased by turning on the LNA and the VGA. Since the PMUT switching section 60 and the wave receiver 70 according to the present embodiment do not turn on the amplifier during the switching period of the number of PMUT cells connected in series at the depth or the time, the power consumption of the amplifier becomes 0 [mW]. Then, the amplifier 81 is turned on at a predetermined depth or time, and the power consumption of the amplifier increases at a depth larger than that. However, the power consumption of the PMUT switching section 60 and the wave receiver 70 of the present embodiment is lower than the power consumption of the configuration of the PMUT switching section, the LNA, and the VGA of the related art. This is because power consumption of the PMUT switching section 60 and the wave receiver 70 of the present embodiment is reduced by VGA of TGC as compared with the conventional configuration.

In addition, with respect to the depth or the time, the ratio of the amplifier ON of the present embodiment PMUT switching section 60 and the wave receiver 70 is lower than the ratio of the amplifier ON in the configuration of the related art. As described above, the PMUT switching section 60 and the wave receiver 70 according to the present embodiment can also reduce the amplifier ON time as compared with the conventional configuration.

As described above, according to the present embodiment, the transmission and reception device 3 includes the plurality of PMUT cells 31 to 33 provided on the CMOS circuit 50, and the CMOS circuit 50. The CMOS circuit 50) includes transmission pulsers 611 to 616 and 641 to 646, series connection switches 621 to 626, and GND switches 631 to 633. The transmission pulsers 611 to 616 and 641 to 646 switch between a high voltage and a low voltage to apply to both sides of the upper electrodes and the lower electrodes of the PMUT cells 31 to 33 at the time of transmission of the ultrasound. The series connection switch 621 to the GND switches 631 to 633 connect the PMUT cells 31 to 33 in series in an arbitrary series number and connect the PMUT cells 31 to 33 between the wave receiver 70 and the common potential at the time of reception of the ultrasound. The common potential is a GND potential.

The transmission and reception device 3 is provided with a drive controller 26 for increasing the number of series connections of the PMUT section corresponding to one reception channel according to the increase of the depth of the ultrasound image. The wave receiver 70 processes a reception signal of the connected PMUT cell. The ultrasonic probe 2 includes a transmission and reception device 3. The ultrasonic diagnostic apparatus 1 includes an ultrasonic probe 2.

The series connection switches 621 to 626 and the GND switches 631 to 633 can switch the number of the PMUT cells 31 to 33 connected in series during the series reception operation. Therefore, when the depth of an ultrasound image is increased, the number of PMUT cells 31 to 33 connected in series can be increased so as to make an adjustment to obtain appropriate element impedance for the wave receiver 70. For example, when the noise becomes equal to the noise floor of the amplifier 82 with low power consumption, the amplifier 82 can be turned on. As illustrated in FIGS. 10 and 11, the amplifier 81 may be turned on. Therefore, since the VGA for TGC is not required, low power consumption can be achieved. Furthermore, since the TGC operation can be achieved by the number of PMUT cells connected in series and turning on/off of the amplifier 82, a high dynamic range can be achieved. In addition, since the VGA can be eliminated, the harmonics performance can be improved by high linearity. By preventing the linearity deterioration, the ultrasound image quality (resolution, diagnosable depth) can be improved.

The PMUT cell 33 close to the wave receiver 70 has a high frequency diaphragm. The PMUT cell 31 far from the wave receiver 70 has a low frequency diaphragm. When the depth of an ultrasound image is small, the drive controller 26 selects a PMUT cell close to the wave receiver 70 and causes the series connection switches 621 to 626 and the GND switches 631 to 633 to connect the PMUT cell. When the depth is great, the drive controller 26 selects a PMUT cell far from the wave receiver 70 and causes the series connection switches 621 to 626 and the GND switches 631 to 633 to connect the selected PMUT cell.

Therefore, the high frequency diaphragm can be connected to the shallow portion where attenuation is small even at a high frequency, and the low frequency diaphragm reaching the deep portion can be connected to the deep portion. Furthermore, providing depth-specific frequency characteristics can increase the effective sensitivity.

### Second Embodiment

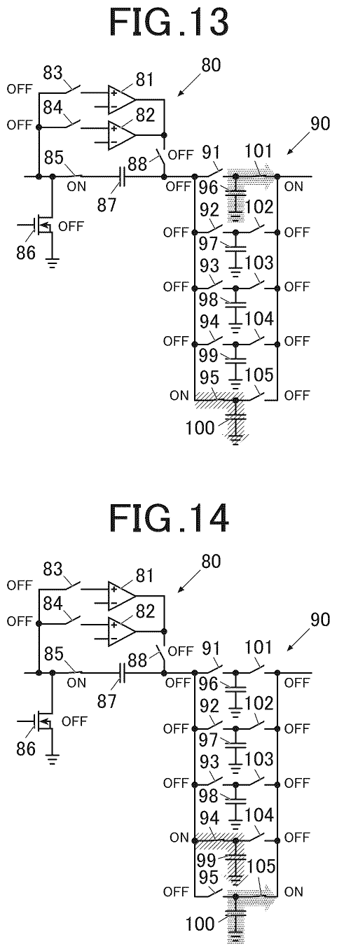

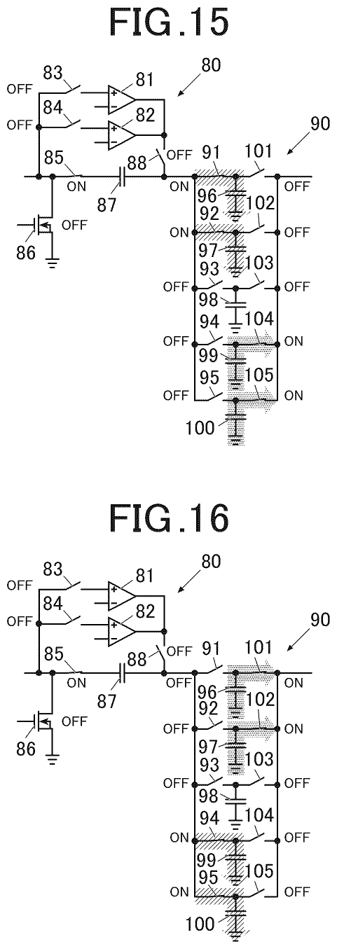

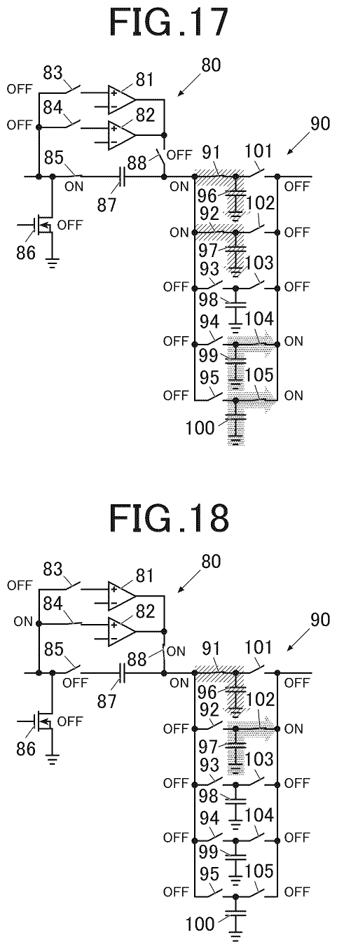

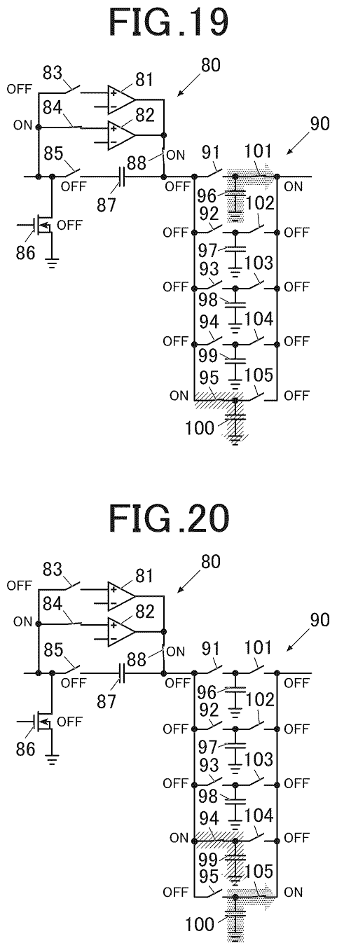

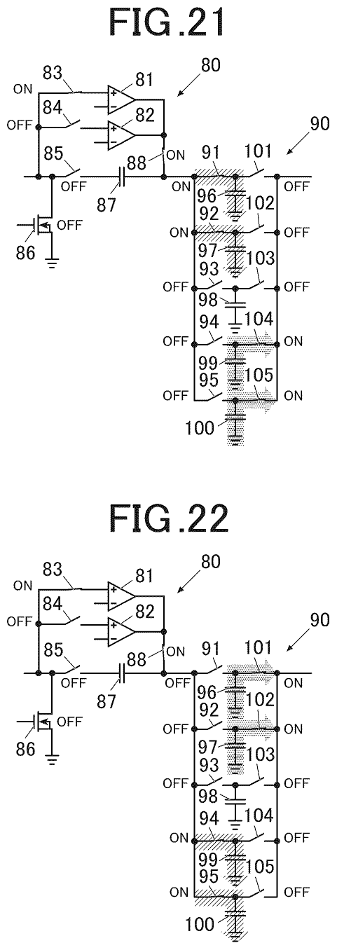

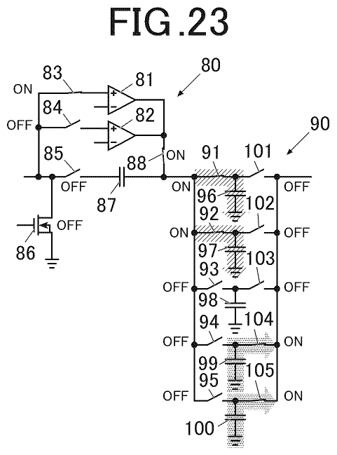

A second embodiment of the present disclosure will be described with reference to FIGS. 12 to 23. FIG. 12 is a diagram illustrating the switching states of the amplification section 80 and the holder 90 of the time phase 0 at the time of the standard amplitude. FIG. 13 is a diagram illustrating a switched state of the amplification section 80 and the holder 90 of a time phase 1 at the time of the standard amplitude. FIG. 14 is a diagram illustrating a switched state of the amplification section 80 and the holder 90 of the time phase 2 at the time of the standard amplitude. FIG. 15 is a diagram illustrating a switching state of the amplification section 80 and the holder 90 of a time phase 0 at the time of ½ amplitude. FIG. 16 is a diagram illustrating a switched state of the amplification section 80 and the holder 90 of the time phase 1 during ½ amplitude. FIG. 17 is a diagram illustrating a switched state of the amplification section 80 and the holder 90 of the time phase 2 at the time of ½ amplitude. FIG. 18 is a diagram illustrating a switch changeover state of the amplification section 80 and the holder 90 in a time phase 0 in a low dynamic range. FIG. 19 is a diagram showing the switching states of the amplification section 80 and the holder 90) in the time phase 1 in the low dynamic range. FIG. 20 is a diagram illustrating a switching state of the amplification section 80 and the holder 90 of the time phase 2 in the low dynamic range. FIG. 21 is a diagram illustrating the switching states of the amplification section 80 and the holder 90 in the time phase 0 in the high dynamic range. FIG. 22 is a diagram illustrating a switching state of the amplification section 80 and the holder 90 of the time phase 1 in the high dynamic range. FIG. 23 is a diagram illustrating switching states of the amplification section 80 and the holder 90 of the time phase 2 at the time of a high dynamic range.

In the first embodiment, the switching operation of the PMUT switching section 60 is performed. In the present embodiment, the switching operation of the wave receiver 70 is performed in addition to the switching operation of the PMUT switching section 60.

The apparatus configuration of the present embodiment uses the ultrasonic diagnostic apparatus 1, similarly to the first embodiment. Therefore, the description of the apparatus is omitted.

Next, operation of the PMUT switching section 60) and the wave receiver 70 of the transmission and reception section 24 of the ultrasonic diagnostic apparatus 1 of the present embodiment will be described with reference to FIG. 12 to FIG. 23. The operation of the PMUT switching section 60 is the same as that of the first embodiment. Here, for example, it is assumed that the reception signal from the PMUT switching section 60 is output to the amplification section 80 at the time of three series ultrasonic wave reception in FIG. 6.

A delay operation in which the amplification section 80 and the holder 90 delay and output a reception signal having a standard amplitude without turning on the amplifiers 81 and 82 will be described with reference to FIGS. 12 to 14. The standard amplitude is the amplitude of a signal corresponding to the capacitance of one capacitor of the holder 90. Here, operations of the amplification section 80 and the holder 90 will be described for each of the time phases 0, 1, and 2 in the order of the three stages in which the operations are different from each other, and the same applies to FIGS. 15 to 23.

As illustrated in FIG. 12, in a time phase 0, the GND switch 86 and the switches 83, 84, and 88 of the amplification section 80 are turned off. The switch 85 is turned on. Therefore, the reception signal from the PMUT switching section 60, is output to the holder 90, via the capacitor 87.

The switches 92, 93, 94, 95, 101, 103, 104, and 105 of the holder 90 are turned off. The switches 91 and 102 are turned on. Therefore, as illustrated by arrows in FIG. 12, the reception signal is charged and held in the capacitor 96. At the same time, the electric charge charged in the capacitor 97 is output as a delayed reception signal having the standard amplitude.

As illustrated in FIG. 13, in a time phase 1 next to the time phase 0 the switches 91 and 102 are newly turned off. The switches 95 and 101 are turned on. Therefore, as illustrated by arrows in FIG. 13, the reception signal is charged and held in the capacitor 100. At the same time, the electric charge charged in the capacitor 96 in the time phase 0 is output as the delayed reception signal of the standard amplitude.

As shown in FIG. 14, in the time phase 2 next to the time phase 1, the switches 95 and 101 are newly turned off. The switches 94 and 105 are turned on. Therefore, as indicated by an arrow in FIG. 14, the reception signal is charged and held in the capacitor 99. At the same time, the electric charge charged in the capacitor 100 in the time phase 1 is output as the delayed reception signal of the standard amplitude.

In this way, charging of one capacitor of the holder 90 and releasing of the charged one capacitor are repeated, and the reception signals with the standard amplitude for one capacitor are delayed and sequentially output.

With reference to FIG. 15 to FIG. 17, descriptions are given of an operation in which the amplification section 80 and the holder 90 output the reception signals with the standard amplitudes of ½ without turning on the amplifiers 81 and 82.

As illustrated in FIG. 15, in the time phase 0, the GND switch 86 and the switches 83, 84, and 88 of the amplification section 80 are turned off. The switch 85 is turned on. Therefore, the reception signal from the PMUT switching section 60 is output to the holder 90 via the capacitor 87.

The switches 93, 94, 95, 101, 102, and 103 of the holder 90 are turned off. The switches 91, 92, 104, and 105 are turned on. Therefore, as indicated by an arrow in FIG. 15, the capacitors 96 and 97 are charged with the reception signal and held. At the same time, the charges charged in the capacitors 99 and 100 are output as the delayed reception signals having the standard amplitudes of ½.

As shown in FIG. 16, in the time phase 1 next to the time phase 0, the switches 91, 92, 104, and 105 are newly turned off. The switches 94, 95, 101, and 102 are turned on. Therefore, as indicated by arrows in FIG. 16, the reception signal is charged and held in the capacitors 99 and 100. At the same time, the charges charged in the capacitors 96 and 97 in the time phase 0 are output as the delayed reception signals with the standard amplitudes of ½.

As shown in FIG. 17, in the time phase 2 next to the time phase 1, the switches 94, 95, 101, and 102 are newly turned off. The switches 91, 92, 104, and 105 are turned on. Therefore, as indicated by an arrow in FIG. 17, the reception signal is charged and held in the capacitors 96 and 97. At the same time, the electric charges charged in the capacitors 99 and 100 in the time phase 1 are output as the delayed reception signals of the standard amplitude of ½.

In this manner, the charging of the two capacitors of the holder 90 and the opening of the two charged capacitors are repeated, and the reception signals with the standard amplitudes of ½ are delayed and sequentially output. The connection impedance is increased by connecting the capacitor 87 having a series capacitance. In addition, it is possible to limit the amplitude of the reception signal to ½ by using the fact that the voltage is lowered by the capacitance division. The above-described delay operation of the reception signal output can realize the phasing of the reception signal.

For example, the drive controller 26 connects the capacitor 87 to the PMUT switching section 60 by switching on the switch 85. Accordingly, the impedance of the PMUT switching section 60 viewed from the delay BF (the AD conversion section and the addition section) can be increased. In the shallow portion of the ultrasound image, the drive controller 26 increases the number of connections of the capacitors 96 to 100 to the PMUT switching section 60 by switching of the switches 91 to 95 to perform the amplitude suppression. In the deep portion, the drive controller 26 reduces the number of connections of the capacitors 96 to 100 to the PMUT switching section 60 by switching of the switches 91 to 95 to increase the amplitude. As the depth increases, the TGC operation can be implemented by outputting reception signals whose amplitudes are sequentially changed to the above-described standard amplitudes of ½, the standard amplitude, and the like. To achieve low power consumption by achieving TGC operation without a VGA.

With reference to FIGS. 18 to 20, an operation in which the amplification section 80 and the holder 90 output the reception signal of the low dynamic range by turning on the amplifier 82 will be described.

As shown in FIG. 18, in the time phase 0, the GND switch 86 and the switches 83 and 85 of the amplification section 80 are turned off. The switches 84 and 88 are turned on. Therefore, the reception signal from the PMUT switching section 60 is output to the holder 90 via the capacitor 87 and the amplifier 82. The amplifier 82 amplifies an input signal and outputs an output signal having a low dynamic range, and has low power consumption.

The switches 92, 93, 94, 95, 101, 103, 104, and 105 of the holder 90 are turned off. The switches 91 and 102 are turned on. Therefore, as illustrated by arrows in FIG. 18, the reception signal is charged and held in the capacitor 96. At the same time, the charge accumulated in the capacitor 97 is output as a delayed reception signal.

As shown in FIG. 19, in the time phase 1 next to the time phase 0 the switches 91 and 102 are newly turned off. The switches 95 and 101 are turned on. Therefore, as indicated by an arrow in FIG. 19, the reception signal is charged and held in the capacitor 100. At the same time, the charge charged in the capacitor 96 at the time phase 0 is output as a delayed reception signal.

As shown in FIG. 20, in the time phase 2 next to the time phase 1, the switches 95 and 101 are newly turned off. The switches 94 and 105 are turned on. Therefore, as indicated by an arrow in FIG. 20, the capacitor 99 is charged with the reception signal and holds the reception signal. At the same time, the electric charge charged in the capacitor 100 in the time phase 1 is output as a delayed reception signal.

As described above, when the required dynamic range of the reception signal is low, the drive controller 26 can connect the amplifier 82 to the PMUT60 by turning on the switch 84. Thus, power consumption can be reduced in a case where a low dynamic range is sufficient in the AD conversion section.

Since the output impedance of the amplifier is smaller than the holding capacitance, the amplitude is not changed, but the magnitude of the noise floor is changed by the magnitude of the holding amount. Therefore, as the number of holding capacitances (capacitors) of the holder 90) connected in parallel decreases, the noise floor also increases. Therefore, when the required dynamic range of the reception signal is low, the number of parallel holding capacitances (capacitors) of the holder 90 can be reduced. Therefore, the driving capability of the amplifier of the amplification section 80 may be low, and the output noise may be considerably large. Therefore, the amplifier 82 with low power consumption can be used.

With reference to FIGS. 21 to 23, an operation in which the amplification section 80 and the holder 90 output the reception signal of the high dynamic range by turning on the amplifier 81 will be described.

As illustrated in FIG. 21, in a time phase 0, the GND switch 86 and the switches 84 and 85 of the amplification section 80 are turned off. The switches 83 and 88 are turned on. Therefore, the reception signal from the PMUT switching section 60 is output to the holder 90 via the capacitor 87 and the amplifier 81. The amplifier 81 amplifies an input signal, outputs an output signal with a high dynamic range, and has high power consumption.

The switches 93, 94, 95, 101, 102, and 103 of the holder 90 are turned off. The switches 91, 92, 104, and 105 are turned on. Therefore, as indicated by arrows in FIG. 21, the reception signals are charged and held in the capacitors 96 and 97. At the same time, the charges accumulated in the capacitors 99 and 100 are output as delayed reception signals.

As shown in FIG. 22, in the time phase 1 next to the time phase 0, the switches 91, 92, 104, and 105 are newly turned off. The switches 94, 95, 101, and 102 are turned on. Therefore, as indicated by arrows in FIG. 22, the reception signal is charged and held in the capacitors 99 and 100. At the same time, the charges charged in the capacitors 96 and 97 at the time phase 0 are output as delayed reception signals.

As shown in FIG. 23, in the time phase 2 next to the time phase 1, the switches 94, 95, 101, and 102 are newly turned off. The switches 91, 92, 104 and 105 are turned on. Therefore, as indicated by arrows in FIG. 23, the reception signal is charged and held in the capacitors 96 and 97. At the same time, the charges charged in the capacitors 99 and 100 in the time phase 1 are output as delayed reception signals.

As described above, when a large dynamic range of the reception signal is required, it is necessary to increase the holding capacitance of the capacitor to be charged in the holder 90 and lower the output noise floor. Therefore, it is necessary to increase the number of capacitors to be charged (to two) and use the amplifier 81 having a high dynamic range.

As illustrated in FIG. 10, the reception signal and the noise of the PMUT switching section 60 and the wave receiver 70 of the present embodiment with respect to the depth or the time are indicated by solid lines. Provided that in FIG. 10 and FIG. 11, the amplification section 80 includes the amplifiers 81 and 82 and an intermediate amplifier (not illustrated). The intermediate amplifier is an amplifier whose power consumption and dynamic range have intermediate values between those of the amplifiers 81 and 82.

When the depth or the time is small, the amplifier of the amplification section 80 is turned off, and the number of series of the PMUT cells in the PMUT switching section 60 is switched from 1 series→2 series→4 series→8 series. At this time, the switch 85 is turned on. Furthermore, the number of capacitors in the holder 90 to be charged and output at the same time was switched at a stage at which the number of series PMUT cells was the same. Specifically, in each of the stages of the switching of the number of series-connected capacitors, charging and outputting are performed simultaneously with three→two→one capacitors in the holder 90 in accordance with an increase in depth or time. With this configuration, amplitude control such as setting the reception signal to 0 [dBV] is facilitated. In particular, in a case where the depth is large, the delay capacitance is left due to the nature of the BF. Therefore, the amplitude can be controlled more easily.

In the PMUT switching section 60 and the wave receiver 70 of the present embodiment, the amplifier is turned on after the switching of the maximum number of series of eight series of the amplifier off, in accordance with an increase in depth or time. In the stage of turning on the amplifier, the amplifier to be turned on is switched to three stages of the amplifier 82→the intermediate amplifier→the amplifier 81 according to the increase of the depth or the time. Therefore, even when the signal level is lowered in accordance with an increase in depth or time, the noise floor can be lowered in three stages. Therefore, the dynamic range of the reception signal to the AD conversion section at the subsequent stage of the PMUT switching section 60 and the wave receiver 70 according to the present embodiment can be increased and noise can be reduced. FIG. 10 illustrates the example in which the amplifier is turned on at the predetermined depth or time, but the present invention is not limited thereto. The amplifier may be turned on at any depth of interest to achieve low noise and a high dynamic range.

The power consumption of the amplifiers of the PMUT switching section 60 and the wave receiver 70 of the present embodiment is shown by a solid line in FIG. 11. Similarly to the first embodiment, the power consumption of the amplifiers of the PMUT switching section 60 and the wave receiver 70 of the present embodiment is 0 [mW] at the amplifier off stage of the depth or the time. Thereafter, in the amplifier-on stage, the power consumption of the amplifier increases in three stages of switching of the amplifier 81→the intermediate amplifier→the amplifier 82 in accordance with an increase in depth or time. Therefore, power consumption of the amplifiers of the PMUT switching section 60 and the wave receiver 70 of the present embodiment is lower than power consumption of the amplifiers of the PMUT switching section 60 and the wave receiver 70 of the first embodiment.

As described above, according to the present embodiment, the transmission and reception device 3 includes the amplification section 80 and the holder 90. The amplification section 80 includes amplifiers 81 and 82 and a capacitor 87 as a series capacitance section. The amplifiers 81 and 82 amplify reception signals at the time of ultrasonic wave reception from the plurality of PMUT cells 31 to 33. The holder 90 includes capacitors 96 to 100 as a plurality of holding capacitance sections that hold the reception signal. The capacitor 87 is provided between the PMUT cells 31 to 33 and the holder 90, and is switchable as a path of the reception signal together with the amplifiers 81 and 82. The capacitance of the capacitor 87 is equal to or smaller than each of the capacitors 96 to 100, or is smaller than the total sum of the capacitors 96 to 100. The drive controller 26 connects the PMUT cells 31 to 33 to the holder 90 via the capacitor 87 in the shallow portion of the ultrasound image, and connects the PMUT cells 31 to 33 to the holder 90 via the amplifiers 81 and 82 in the deep portion.

Therefore, in the shallow portion of the ultrasound image, by connecting the capacitor 87, the impedance of the PMUT switching section 60 viewed from the delay BF (the AD conversion section and the addition section) can be increased, and in the deep portion, switching to the amplifiers 81 and 82 can be performed. Therefore, since the VGA for TGC is not required, low power consumption can be achieved. Furthermore, since the TGC operation can be achieved by switching between the capacitor 87 and the amplifiers 81 and 82, a high dynamic range can be achieved. Furthermore, an increase in the area occupied by the holder 90 on a semiconductor is suppressed by diverting an existing function component (delay BF holding capacitance) to the holder 90. Furthermore, the wave receiver 70) and the PMUT switching section 60 as a high-linearity wave transmission circuit can be mixedly mounted, and harmonics performance can be improved.

Furthermore, the capacitance of the capacitor 87 is equal to or smaller than the input capacitance of the amplifiers 81 and 82. Therefore, amplitude variation at the time of switching between the amplifiers 81 and 82 and the capacitor 87 can be suppressed. In addition, from the viewpoint of prevention of S/N deterioration, it is desirable that the input capacitance of the capacitor 87 and the amplifiers 81 and 82 is the same as the capacitance when the PMUT cells of the PMUT switching section 60 are controlled to be in maximum series.

The drive controller 26 connects the capacitor 87 to the PMUT cells 31 to 33 and increases the number of connections of the capacitors 96 to 100 in the shallow portion of the ultrasound image to perform the amplitude suppression. The drive controller 26 reduces the number of connections of the capacitors 96 to 100 to the PMUT cells 31 to 33 to increase the amplitude in the deep portion. Therefore, the TGC operation can be implemented without a VGA, and low power consumption can be implemented.

The amplifiers 81 and 82 are a plurality of amplifiers having different power consumption and dynamic ranges. The drive controller 26 causes the amplifier 82 with low power consumption and a low dynamic range to amplify the reception signal in the shallow portion of the ultrasound image. The drive controller 26 causes the amplifier 81 with high power consumption and a high dynamic range to amplify the reception signal at the deep portion (or the depth of interest). Therefore, both a high dynamic range and low power consumption can be achieved.

### First Modification Example

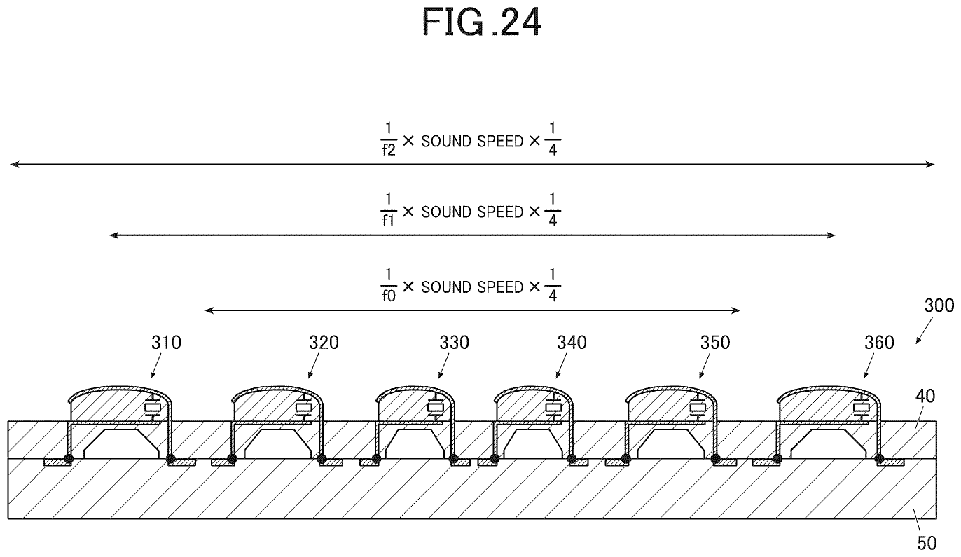

A first modification example of the above-described first embodiment will be described with reference to FIG. 24. FIG. 24 is a schematic cross-sectional view showing a PMUT array 300 and a CMOS circuit 50.

In the first embodiment, the PMUT array 21 of the ultrasonic probe 2 has a configuration in which the shapes of the PMUT cells 31 to 33 are the same as shown in the PMUT array 30 of FIG. 2. Therefore, characteristics such as frequency of the PMUT cells 31 to 33 are also the same. The present modification example is a configuration in which the frequencies of the PMUT cells of the PMUT array are made different.

As with the above-described first embodiment, the ultrasonic diagnostic apparatus 1 is used as the apparatus configuration of the present modification example. However, the PMUT array 300 on the CMOS circuit 50 shown in FIG. 24 is used as the PMUT array 21 of the ultrasonic probe 2. The PMUT array 300 includes PMUT cells 310, 320, 330, 340, 350, and 360 as the PMUT cells 211. The PMUT cells 310 to 360 illustrate the PMUT cell 211 as a representative, and are not limited to this number.

The PMUT cells 310 to 360 are arranged in the arrangement order of FIG. 24 in the elevation direction of the transmission and reception opening of the PMUT cell 211 of the ultrasonic probe 2. It is assumed that the PMUT cells 330 and 340 are arranged at the center in the elevation direction of the transmission and reception opening. It is assumed that the PMUT cells 320 and 350 are arranged at an intermediate position between the center and the end of the transmission and reception opening in the elevation direction. It is assumed that the PMUT cells 310 and 360 are arranged at end portions of the transmission and reception opening in the elevation direction.

The PMUT cells 310 to 360 have the same structure as the PMUT cells 31 to 33 of FIG. 2, but have shapes in which frequencies of ultrasound to be transmitted and received are different. Specifically, the diameters of the PMUT diaphragms of the PMUT cells 310 to 360 are different. The larger the diameter, the lower the resonant (center) frequency. The (resonance) frequency corresponds to an ultrasound to be transmitted or received. In the PMUT cells 330 and 340, the diameters of the PMUT diaphragms are large, and the frequencies thereof are referred to as frequencies f0. The PMUT cells 320 and 350 have intermediate diameters of the PMUT diaphragms and have the frequencies thereof as a f1 of frequencies. The PMUT cells 310 and 360 have small-diameter PMUT diaphragms and have frequencies thereof as a f2 of frequencies. The frequencies have a relationship of f0>f1>f2.

As shown in FIG. 24, the diameters of the respective PMUT diaphragms of the PMUT cells 310 to 360 are shorter than ¼ wavelength (=1/f×sound speed×¼) of the ultrasound in the medium. Therefore, the sound pressures applied to the adjacent PMUT diaphragms can be regarded as equal. In addition, the output voltage can be increased by connecting the PMUT cells in series. It also increases the frequency band of the PMUT diaphragm total. Therefore, each of the PMUT cells 310 and 360, the PMUT cells 320 and 350, and the PMUT cells 330) and 340 is provided with a plurality of center frequencies different from each other.

At this time, the PMUT cells 330 and 340 of the high frequency diaphragm are preferably placed at the center in the elevation direction in the transmission and reception opening of the PMUT cell 211. This is because the high frequency diaphragm attenuates more closely in consideration of attenuation of an acoustic medium. In addition, for convenience of connecting the PMUT cells in a row, which is a feature of the present disclosure, it is desirable that the PMUT cells 330 and 340 of the high frequency diaphragm are first connected to the wave receiver 70. That is, the PMUT cells 330 and 340 at the center of the transmission and reception opening in the elevation direction and close to the wave receiver 70 have a high-frequency (frequency-side f0) diaphragm. PMUT cells 320 and 350 located at intermediate positions in the elevation direction in the transmission and reception openings and at intermediate positions from the wave receiver 70 have diaphragms for intermediate frequencies (f1 frequencies). The PMUT cells 310 and 360 located at the end of the transmission and reception opening in the elevation direction and far from the wave receiver 70 include a low-frequency (f2) diaphragm.

As described above, according to the present modification example, the PMUT cell close to the wave receiver 70 has the high frequency diaphragm and is arranged at a position close to the center of the transmission and reception opening in the elevation direction. The PMUT cell far from the wave receiver is disposed at a position close to the outside of the transmission and reception opening in the elevation direction. Therefore, it is possible to provide an aperture synthesis effect for each frequency. Furthermore, high-frequency ultrasound with large attenuation in the depth direction can be transmitted and received at the center of the transmission and reception opening, and low-frequency ultrasound with small attenuation in the depth direction can be transmitted and received outside the transmission and reception opening.

### Second Modification Example

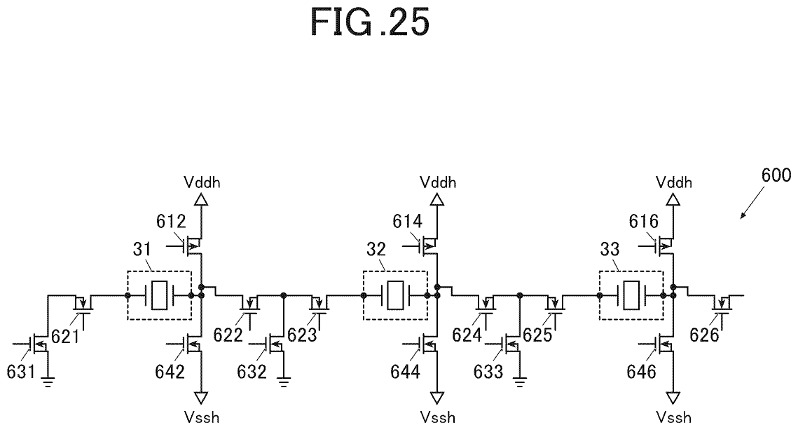

Referring to FIG. 25, a second modification example of the first embodiment described above will be described. FIG. 25 is a circuit diagram illustrating a PMUT switching section 600 of the present modification example.

In the first embodiment, the PMUT switching section 60 of the CMOS circuit 50 has a full-bridge circuit configuration with respect to the PMUT cells 31 to 33. In the present modification example, the PMUT switching section has a circuit configuration of a half bridge with respect to the PMUT cell.

As with the above-described first embodiment, the ultrasonic diagnostic apparatus 1 is used as the apparatus configuration of the present modification example. However, the PMUT switching section 600 illustrated in FIG. 25 is used as the PMUT switching section 60 of the CMOS circuit 50. The PMUT switching section 600 includes PMUT cells 31 to 33, transmission pulsers 612, 614, 616, 642, 644, and 646, series connection switches 621 to 626, and GND switches 631 to 633.

The PMUT switching section 600 cannot take a drive path for the parallel positive sound pressure of FIG. 4 and the parallel negative sound pressure of FIG. 5 at the time of ultrasound transmission with respect to the PMUT cells 31 to 33. For example, the transmission pulser 612, the series connection switch 621, and the GND switch 631 are turned on for the PMUT cell 31. The transmission pulser 642 and the series connection switch 622 are turned off. Thus, a positive voltage between the high voltage Vddh and the GND potential is applied to the PMUT cell 31, and the PMUT cell 31 is driven with a positive acoustic pressure.

For the PMUT cell 31, the transmission pulser 642, the series connection switch 621, and the GND switch 631 are turned on. The transmission pulser 612 and the series connection switch 622 are turned off. Accordingly, a voltage between the low voltage Vssh and the GND potential is applied to the PMUT cell 31, and the PMUT cell 31 is driven by a negative sound pressure. Ultrasound is transmitted from the PMUT cell 31 by repeating the driving of the positive sound pressure and the negative sound pressure of the PMUT cell 31. Driving of the PMUT cells 32 and 33 is similar to driving of the PMUT cell 31.

The PMUT switching section 600 cannot apply the voltage of high voltage Vddh-low voltage Vssh to the PMUT cells 31 to 33. Therefore, the high voltage Vddh corresponding to the PMUT switching section 600 needs to be twice as high as the high voltage Vddh corresponding to the PMUT switching section 60 of the embodiment. Furthermore, the low voltage Vssh corresponding to the PMUT switching section 600 needs to be twice as low as the low voltage Vssh corresponding to the PMUT switching section 60.

As described above, according to the present modification example, the transmission and reception device 3 includes the plurality of PMUT cells 31 to 33 provided on the CMOS circuit 50, and the CMOS circuit 50. The CMOS circuit 50 includes transmission pulsers 612, 614, 616, 642, 644, and 646, series connection switches 621 to 626, and GND switches 631 to 633. The transmission pulsers 612 to 646 switch between a high voltage and a low voltage to apply to one side of the upper electrodes and the lower electrodes of the PMUT cells 31 to 33 at the time of transmission of ultrasound. Therefore, it is possible to achieve the same effects as those of the transmission and reception device 3 of the first embodiment and simplify the configuration of the CMOS circuit 50 (PMUT switching section 600).

### Third Modification Example

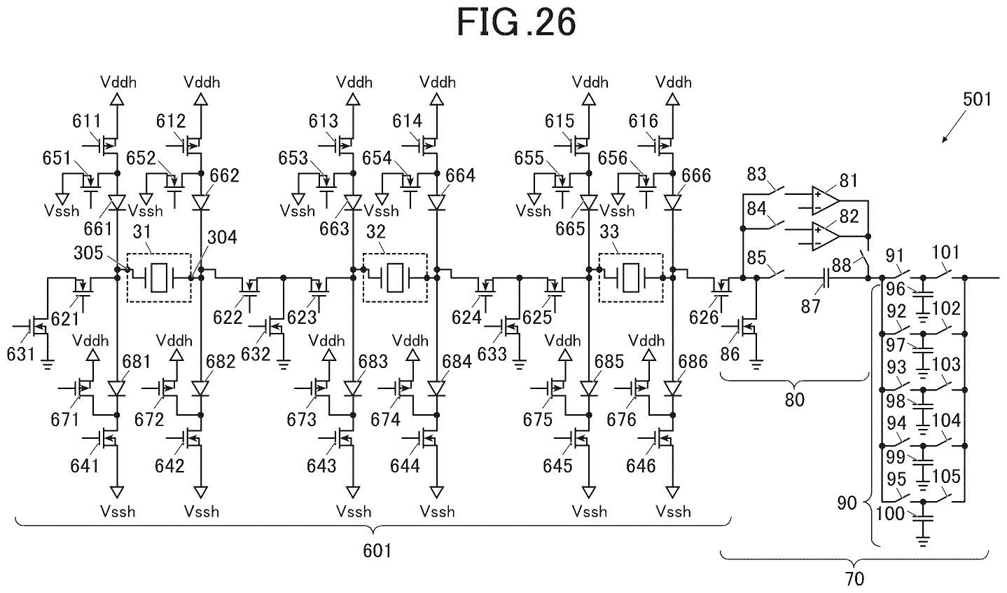

A third modification example of the above-described first embodiment will be described with reference to FIG. 26. FIG. 26 is a circuit diagram illustrating the PMUT switching section 601 and the wave receiver 70 of the CMOS circuit 501 of the present modification example.

In the first embodiment described above, in the PMUT switching section 60 of the CMOS circuit 50 of FIG. 3, the transmission pulsers 611 to 616 and 641 to 646 are turned off during reception of an ultrasonic wave. However, when a transistor with a higher withstand voltage is used for the transmission pulsers 611 to 616 and 641 to 646 in order to apply a pulse with a higher voltage, the off-capacitance further increases. Therefore, at the time of reception, these become a dominant term as parasitic capacitance, and it is difficult to obtain the effect of serialization. The present modification example provides a PMUT switching section that improves such matters.

As the apparatus configuration of the present modification example, the ultrasonic diagnostic apparatus 1 is used as in the above-described embodiment. However, the CMOS circuit 50 is replaced by a CMOS circuit 501 shown in FIG. 26. The CMOS circuit 501 includes the PMUT switching section 601 illustrated in FIG. 26 and the wave receiver 70. The PMUT switching section 601 includes the PMUT cells 31 to 33, the transmission pulsers 611 to 616 and 641 to 646, the series connection switches 621 to 626, the GND switches 631 to 633, the reverse bias switches 651 to 656 and 671 to 676, and the diodes 661 to 666 and 681 to 686. The reverse bias switches 651 to 656 and 671 to 676 function as a reverse bias section. The transmission pulsers 611 to 616 and 641 to 646 are high-withstand-voltage MOSFETs.

Here, circuit elements related to the PMUT cell 31 will be representatively described. Provided that circuit elements described in the embodiment are denoted by the same reference signs, and description thereof is omitted. The reverse bias switch 651 is an NMOSFET whose drain is connected to the drain of the transmission pulser 611 and whose source is connected to the power supply of the low voltage Vssh. The reverse bias switch 652 is an NMOSFET whose drain is connected to the drain of the transmission pulser 612 and whose source is connected to the power supply of the low voltage Vssh. The diode 661 is a PN junction diode whose anode is connected to the drains of the transmission pulser 611 and the reverse bias switch 651 and whose cathode is connected to the TSV305. The diode 662 is a PN junction diode whose anode is connected to the drains of the transmission pulser 612 and the reverse bias switch 652 and whose cathode is connected to the TSV304.

The reverse bias switch 671 is a PMOSFET whose drain is connected to the drain of the transmission pulser 641 and whose source is connected to the power supply of the high voltage Vddh. The reverse bias switch 672 is a PMOSFET whose drain is connected to the drain of the transmission pulser 642 and whose source is connected to a power supply of a high voltage Vddh. The diode 681 is a PN junction diode whose anode is connected to a TSV305 and whose cathode is connected to the drains of the transmission pulser 641 and the reverse bias switch 671. The diode 682 is a PN junction diode whose anode is connected to a TSV304 and whose cathode is connected to the drains of the transmission pulser 642 and the reverse bias switch 672.

The reverse bias switches 653, 654, 673, and 674 related to the PMUT cell 32 are similar to the reverse bias switches 651, 652, 671, and 672 related to the PMUT cell 31, respectively. The diodes 663, 664, 683, and 684 related to the PMUT cell 32 are similar to the diodes 661, 662, 681, and 682 related to the PMUT cell 31, respectively.

The reverse bias switches 655, 656, 675, and 676 related to the PMUT cell 33 are similar to the reverse bias switches 651, 652, 671, and 672 related to the PMUT cell 31, respectively. The diodes 665, 666, 685, and 686 related to the PMUT cell 33 are similar to the diodes 661, 662, 681, and 682 related to the PMUT cell 31, respectively.

In the reverse bias switches 651 to 656 and 671 to 676, a drive signal from the drive controller 26 is input to each gate, and switching control is performed. The reverse bias switch 651 and the diode 661 function as the separator 602. In this way, a combination of the diode and the reverse bias switch connected to the same transmission pulser, such as the reverse bias switch 652 and the diode 662, is set as the separator 602.

When ultrasound is transmitted, the gates of the reverse bias switches 651 to 656 and 671 to 676 are turned off. In addition, the operation of other circuit elements of the PMUT switching section 601 other than the separator 602 in the transmission is the same as the operation of other circuit elements of the PMUT switching section 600.

When switching from transmission to reception of ultrasound, the gates of the reverse bias switches 651 to 656 and 671 to 676 are turned on. When the gates are turned on the diodes 661 to 666 and 681 to 686 are reversely biased. Thus, the off-capacitance of the transmission pulsers 611 to 616 and 641 to 646 can be separated from the PMUT cells 31 to 33.

The separation utilizes the fact that the on-resistance/off-capacitance ratio of a diode is lower than the on-resistance/off-capacitance ratio of a unipolar FET. Here, a circuit configuration in which the diodes 661 to 666 and 681 to 686 of the PMUT switching section 601 are replaced with body diodes of FETs will be considered. In the circuit configuration and the PMUT switching section 601, the capacitance in the off state has the same structure, and thus the FET and the diode have substantially the same capacitance. On the other hand, in the on-state, the diode has no channel resistance unlike the FET, so that it is possible to make the on-resistance lower than the FET. Therefore, the ratio of on-resistance/off-capacitance is lower in the diode than in the FET. Also, a normal PN junction diode requires a “reverse recovery time” during which it is slow to turn off. However, a problem is a minimum TOF (Time Of Flight) in which an ultrasound is transmitted and returned. Therefore, since the reverse recovery time of the diodes 661 to 666 and 681 to 686 of the PN junction is 1 [μs] or less, there is no problem in reception of the ultrasound.

As described above, when ultrasound is received, the gates of the reverse bias switches 651 to 656 and 671 to 676 are turned on. Since the reverse bias switches 651 to 656 and 671 to 676 only supply the reverse bias voltage, transistors having a minimum size allowed by a process rule can be used. Therefore, the off-capacitance of the reverse bias switches 651 to 656 and 671 to 676 can be made sufficiently small. Furthermore, the operation of the other circuit elements of the PMUT switching section 601 in reception is similar to the operation of the other circuit elements of the PMUT switching section 60. The separator 602 of the PMUT switching section 601 is a device configuration which is not necessary in principle but is actually necessary.

As described above, according to the present modification example, the CMOS circuit 501 includes the PMUT switching section 601 and the wave receiver 70. The PMUT switching section 601 includes a separator 602. The separator 602 separates off the off-capacitance of the transmission pulsers 611 to 616 and 641 to 646 from the PMUT cells 31 to 33 at the time of reception of ultrasound. The separator 602 includes diodes 661 to 666 and 681 to 686 and reverse-biased switches 651 to 656 and 671 to 676. The diodes 661 to 666 and 681 to 686 are connected to the transmission pulsers 611 to 616 and 641 to 646 and the PMUT cells 31 to 33. The reverse bias switches 651 to 656 and 671 to 676 set the diodes 661 to 666 and 681 to 686 in a reverse bias state at the time of reception. Therefore, during reception of ultrasound, the off-capacitance of the transmission pulsers 611 to 616 and 641 to 646 can be disconnected from the PMUT cells 31 to 33. Therefore, it is possible to reliably obtain the effect of the serialization of the PMUT cells 31 to 33 at the time of reception.

Note that the description in the above embodiment and modification example is an example of the transmission and reception device, the ultrasonic probe, and the ultrasonic diagnostic apparatus according to the present disclosure and is not limited thereto. For example, at least two of the embodiment and modification example may be combined as appropriate.

For example, although the amplification section 80 is configured to have the two amplifiers 81 and 82 different in power consumption and dynamic range in the embodiment and the modification example described above, it is not limited to this. For example, as described in FIGS. 10 and 11, the amplification section 80 may be configured to include three or more amplifiers having different power consumptions and dynamic ranges. Furthermore, the amplification section 80 may be configured to include one amplifier whose power consumption and dynamic range are switchable.

Although embodiments of the present invention have been described and shown in detail, the disclosed embodiments are made for purposes of illustration and example only and not limitation. The scope of the present invention should be interpreted by terms of the appended claims.

According to the above feature, low power consumption and a high dynamic range can be achieved in the TGC.

What is claimed is:

1. A transmission and reception device comprising:
a plurality of PMUT sections in which each PMUT section includes one or more PMUTs on a CMOS circuit; and
the CMOS circuit, wherein
the CMOS circuit includes:
a wave transmitter that switches a voltage between a high voltage and a low voltage and applies the switched voltage to one or both of an upper electrode and a lower electrode of the PMUT section at a time of transmission of an ultrasound; and
a series connector that connects the PMUT sections in series in an arbitrary number of series and connects the connected PMUT sections between a wave receiver and a common potential at a time of reception of an ultrasound, the wave receiver processing a reception signal of the connected PMUT sections.

2. The transmission and reception device according to claim 1, further comprising a hardware processor that increases the number of series of connection of the PMUT sections corresponding to one reception channel in response to an increase in depth of an ultrasound image.

3. The transmission and reception device according to claim 1, wherein
the PMUT section close to the wave receiver includes a high frequency diaphragm, and
the PMUT section far from the wave receiver includes a low frequency diaphragm.

4. The transmission and reception device according to claim 3, further comprising a hardware processor that, when a depth of an ultrasound image is small, selects the PMUT section close to the wave receiver and causes the series connector to connect the selected PMUT section and, when the depth is large, selects the PMUT section far from the wave receiver and causes the series connector to connect the selected PMUT section.

5. The transmission and reception device according to claim 3, wherein
the PMUT section close to the wave receiver is arranged at a position close to a center of a transmission and reception opening in an elevation direction, and
the PMUT section far from the wave receiver is arranged at a position close to an outer side of the transmission and reception opening in the elevation direction.

6. The transmission and reception device according to claim 1, further comprising a separator that separates the wave transmitter from the PMUT section at a time of reception of an ultrasound.

7. The transmission and reception device according to claim 6, wherein
the separator includes:
a diode connected to the wave transmitter and the PMUT section; and
a reverse bias section that causes the diode to be in a reverse bias state at the time of the reception.

8. A transmission and reception device comprising:
an amplifier section configured to amplify a reception signal at a time of ultrasound reception from a PMUT section including one or a plurality of PMUTs;
a holder including a plurality of holding capacitance sections for holding the reception signal; and
a series capacitance section provided between the PMUT section and the holder and switchable together with the amplifier section as a path of the reception signal, wherein
a capacitance of the series capacitance section is smaller than or equal to a capacitance of each of the holding capacitance sections or is smaller than a total sum of capacities of the plurality of holding capacitance sections.

9. The transmission and reception device according to claim 8, further comprising a hardware processor that connects the PMUT section to the holder via the series capacitance section in a shallow portion of an ultrasound image and connects the PMUT section to the holder via the amplifier section in a deep portion.

10. The transmission and reception device according to claim 8, wherein a capacitance of the series capacitance section is equal to or smaller than an input capacitance of the amplifier section.

11. The transmission and reception device according to claim 8, further comprising a hardware processor that connects the series capacitance section to the PMUT section, performs amplitude suppression by increasing a number of connection of the holding capacitance sections in a shallow portion of an ultrasound image, and performs amplitude increase by decreasing the number of the connection of the holding capacitance sections to the PMUT section in a deep portion.

12. The transmission and reception device according to claim 8, further comprising a hardware processor, wherein
the amplifier section includes a plurality of amplifiers having different power consumptions and dynamic ranges or an amplifier whose power consumption and dynamic range are switchable, and
the hardware processor causes the amplifier with a low power consumption and a low dynamic range to amplify the reception signal in a shallow portion of an ultrasound image and causes the amplifier with a high power consumption and a high dynamic range to amplify the reception signal in a deep portion or a depth of interest.

13. An ultrasonic probe comprising the transmission and reception device according to claim 1.

14. An ultrasonic diagnostic apparatus comprising the ultrasonic probe according to claim 13.

15. An ultrasonic probe comprising the transmission and reception device according to claim 8.

16. An ultrasonic diagnostic apparatus comprising the ultrasonic probe according to claim 15.
